(19) United States
(12) Patent Application Publication
(10) Pub. No.: US 2026/0151339 A1
(43) Pub. Date: Jun. 4, 2026

(54) GLUTATHIONE-RESPONSIVE METHOTREXATE POLYMERSOMES FOR MANAGEMENT OF ECTOPIC PREGNANCY

(71) Applicants: Oregon State University, Corvallis, OR (US); Oregon Health & Science University, Portland, OR (US)

(72) Inventors: Olena Taratula, Corvallis, OR (US); Oleh Taratula, Corvallis, OR (US); Maureen Baldwin, Portland, OR (US); Leslie Myatt, Portland, OR (US); Babak Mamnoon, Corvallis, OR (US)

(73) Assignees: Oregon State University, Corvallis, OR (US); Oregon Health & Science University, Portland, OR (US)

(21) Appl. No.: 19/439,246

(22) Filed: Jan. 2, 2026

Related U.S. Application Data
(63) Continuation-in-part of application No. PCT/US2024/036825, filed on Jul. 3, 2024.
(60) Provisional application No. 63/512,003, filed on Jul. 5, 2023.

(51) Int. Cl. A61K 9/1273; A61K 9/00; A61K 31/519; A61K 47/34; A61K 47/54; A61K 47/69; A61P 15/00; A61P 35/00

(52) U.S. Cl. CPC A61K 9/1273; A61K 9/0019; A61K 31/519; A61K 47/34; A61K 47/549; A61K 47/555; A61K 47/6915; A61P 15/00; A61P 35/00

(57) ABSTRACT

Methotrexate-containing polymersomes and methods for treating ectopic pregnancy or for treating gestational choriocarcinoma using the methotrexate-containing polymersomes.

### CROSS-REFERENCE TO RELATED APPLICATION

This application is a continuation-in-part of International Application No. PCT/US2024/036825, filed Jul. 3, 2024, which claims the benefit of U.S. Patent Application No. 63/512,003, filed Jul. 5, 2023, the disclosure of each of which is expressly incorporated herein by reference in its entirety.

### STATEMENT OF GOVERNMENT LICENSE RIGHTS

This invention was made with government support under R01CA237569, R01HD101450, R03TR004020, and R37CA234006, each awarded by the National Institutes of Health. The government has certain rights in the invention.

### BACKGROUND

The first-line treatment for ectopic pregnancy (EP), the chemotherapeutic methotrexate (MTX), has a failure rate of more than 10%, which can lead to severe complications or death. Inadequate accumulation of administered MTX at the ectopic implantation site significantly contributes to therapeutic failure.

Ectopic pregnancy (EP) is defined as the abnormal implantation of an embryo, most often outside of the uterus, and accounts for about 1-2% of pregnancies in the USA. Hemorrhage caused by ruptured EP continues to be the primary cause of first-trimester maternal death, accounting for 16% of first-trimester emergency room visits annually. EP is presumed when serum human chorionic gonadotropin (hCG) levels surpass 3,000 mIU mL-1 and there is no intrauterine gestational sac visible by ultrasound, and if hCG levels do not decline after uterine evacuation. The most common treatment of confirmed or presumed early unruptured EP is systemic administration of the chemotherapy agent methotrexate (MTX), an inhibitor of folate-dependent steps in nucleic acid synthesis, which effectively destroys the rapidly dividing ectopic trophoblast and thus prevents the placenta from developing and invading adjacent tissues. The recommended clinical dosing regimen for MTX is a single intramuscular injection of 1 mg kg-1 or 50 mg m-2. Unfortunately, the failure rate of MTX treatment can exceed 10%; the risk factors for which are poorly understood but may include high body mass index, rapid clearance, or inaccurate diagnosis. Rapid clearance of MTX most likely leads to insufficient accumulation of the drug at the ectopic implantation site, which ultimately results in therapeutic failure. In this scenario, repeated or higher doses are required, which can result in various side effects ranging from nausea and vomiting to interstitial pneumonitis and bone marrow suppression. As MTX remains the first-line treatment for EP, there is a pressing need to improve MTX efficacy. MTX treatment is also associated with substantial side effects.

Despite the advances in treating EP with MTX noted above, a need exists for MTX formulations that improve the delivery and retention of an effective dose of MTX in the developing placenta, the site of ectopic implantation and, consequently, increase its safety and efficacy to provide enhanced therapeutic outcomes. The present disclosure seeks to fulfill this need and provides further related advantages.

### SUMMARY

In one aspect, the present disclosure provides a methotrexate-containing polymersome. In certain embodiments, methotrexate-containing polymersome comprises:
(a) methotrexate, or a pharmaceutically acceptable salt thereof, and
(b) an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block, and
wherein methotrexate, or a pharmaceutically acceptable salt thereof, is encapsulated in the hydrophilic interior core.

In certain embodiments, the polymersome's hydrophilic PEG block has a molecule weight of about 2 kDa and the hydrophobic PCL block has a molecule weight of about 5 kDa.

In one related aspect, the disclosure provides pharmaceutical compositions comprising the polymersome described herein and a pharmaceutically acceptable carrier.

In another related aspect, the disclosure provides the polymersomes described herein in lyophilized form.

In further aspect, the present disclosure provides a method for treating an ectopic pregnancy. In certain embodiments, the methods comprise administering a therapeutically effective amount of a methotrexate-containing polymersome to a subject in need thereof. In the method, the amount of methotrexate, or a pharmaceutically acceptable salt thereof, is sufficient to result in resolution of serum human chorionic gonadotropin. In certain embodiments, the methotrexate-containing polymersome is administered in a single dose. In other embodiments, the methotrexate-containing polymersome is administered in two doses.

In another aspect, the present disclosure provides a method for treating gestational trophoblastic neoplasia (GTN) in a subject, comprising administering to a subject in need thereof, a therapeutically effective amount of a methotrexate-containing polymersome, as described herein, wherein the polymersome comprises one or more moieties to target equilibrative nucleoside transporter 1 (ENT-1) expressed on the surface of the neoplasia cells.

In a related aspect, the present disclosure provides a method for treating gestational choriocarcinoma in a subject, comprising administering to a subject in need thereof, a therapeutically effective amount of a methotrexate-containing polymersome, as described herein, wherein the polymersome comprises one or more guanosine moieties to target equilibrative nucleoside transporter 1 (ENT-1) expressed on the surface of the choriocarcinoma cells.

In certain embodiments of the methods described herein, the subject is a human subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1L presents schematic illustrations and the characterization of representative methotrexate-containing polymersomes of the disclosure. Schematic illustrations of MTX-SS-Ps (PEG-PCL disulfide bond-containing polymersome loaded with MTX) (FIG. 1A), MTX-Ps (polymersome lacking disulfide bond and loaded with MTX) (FIG. 1B), and NIR-SS-Ps (disulfide bond-containing polymersome loaded with NIR dye (silicon naphthalocyanine) (FIG. 1C). Size distribution measured with dynamic light scattering (DLS) for MTX-SS-Ps (38.2 nm±0.4, PDI: 0.11, n=3) (FIG. 1D), MTX-Ps (35.5 nm±0.3, PDI: 0.10, n=3) (FIG. 1E), and NIR-SS-Ps (39.3 nm±0.5, PDI: 0.12, n=3) (FIG. 1F). Cryo-TEM images of MTX-SS-Ps (FIG. 1G) and MTX-Ps (FIG. 1H). Stability study via DLS size measurements for MTX-SS-Ps and MTX-Ps over 8 weeks (FIG. 1I). Drug release profiles of MTX-SS-Ps in PBS (pH=7.4) buffer containing 5 μM, 1 mM, and 10 mM GSH (FIG. 1J), and MTX-SS-Ps and MTX-Ps at pH 7.4 without GSH (FIG. 1K) and with 10 mM GSH (FIG. 1L).

FIGS. 2A-2E illustrate biodistribution of NIR polymersomes in pregnant mice at Gd7.5. Fluorescence images of major organs (FIG. 2A) and mouse uteri (FIG. 2B), resected from pregnant mice (n=3) 24 hours post-injection (i.v.) at Gd6.5 with saline (control), NIR-Ps (0.3 mg mL-1 dye, 100 μL) and NIR-SS-Ps (0.3 mg mL-1 dye, 100 μL). Semiquantitative analysis of fluorescence signal in 5 organs and uteri at Gd7.5, 24 hours post-injection with NIR-Ps, NIR-SS-Ps and saline (control) (FIG. 2C). Values are expressed as mean±SD (n=3). *P<0.05, **P<0.01, ***P<0.001, ****P<0.0001. H&E stained and corresponding fluorescence images of thin sections acquired at ex./em. 710/775 nm of fetoplacental units from mice injected at Gd6.5 and collected 24 hours post-injection with NIR-Ps (0.3 mg mL-1 dye, 100 μL) (FIG. 2D) and NIR-SS-Ps (0.3 mg mL-1 dye, 100 μL) (FIG. 2E). Scale bar=1 mm. am—antimesometrial decidua, md—mesometrial decidua, em—embryo, epc—ectoplacental cone, i.e.—implantation crypt, where em, epc, and i.e. are outlined in black.

FIGS. 3A-3F illustrate photoacoustic imaging of placenta at Gd13.5 with NIR polymersomes. Photoacoustic (PA) signal overlaid with ultrasound (US) images of ex vivo fetoplacental units, depicting NIR dye and oxygenated hemoglobin, excised from pregnant mice (n=3) 24 hours following i.v. injection with saline (control) (FIG. 3A) and NIR-SS-Ps (0.3 mg mL-1 NIR dye, 100 μL) (FIG. 3B) at Gd12.5 (Vevo LAZR imaging system equipped with an LZ550 transducer). Scale bar=1 mm. PA signal spectra (averaged) corresponding to placental and fetal tissues from FIGS. 2A and 3B in pregnant mice treated with saline (FIG. 3C) and NIR-SS-Ps (FIG. 3D). H&E (FIG. 3E) and corresponding fluorescence images (FIG. 3F) of thin section acquired at ex./em. 710/775 nm of a representative fetoplacental unit from mice injected with NIR-SS-Ps at Gd12.5 and collected 24 hours post-injection. Scale bar=1 mm. a.c.—amniotic cavity, am—amnion, c.p.—chorionic plate, dec—decidua, em—embryo, j.z.—junctional zone, lab—labyrinth, mt—metrial triangle, um—umbilicus.

FIGS. 4A-4C illustrate embryonic development following administration of MTX-SS-Ps and controls. Schematic timeline of treatment and imaging: MTX-SS-Ps (1 mg kg-1) was i.v. injected at Gd6.5 and Gd8.5, concurrent with and followed by US monitoring through Gd13.5 (n=3) (FIG. 4A). Gestational sac length measurements acquired during US imaging. Values are expressed as mean±SD (n=3). **P<0.01, ***P<0.001 (FIG. 4B). US images of embryos from each treatment group; MTX-SS-Ps and controls (saline, empty Ps, empty SS-Ps, free MTX and MTX-Ps (1 mg kg-1 MTX at Gd6.5 and Gd8.5) (FIG. 4C). Scale bar=1 mm.

FIGS. 5A and 5B illustrate ex vivo evaluation of murine pregnancy progress following i.v. administration of free MTX, MTX-SS-Ps, MTX-Ps, and controls (saline and empty polymersomes). Photographs of (A) exteriorized uteri (FIG. 5A) and resected uteri (FIG. 5B) of mice (n=3) from FIGS. 4A-4C, euthanized at Gd13.5.

FIGS. 6A-6D illustrate histological assessment of MTX nanomedicine treatment. Top panels: H&E-stained thin sections of selected fetoplacental units from FIG. 5B: saline (FIG. 6A), free MTX (FIG. 6B), and MTX-Ps (FIG. 6C), and non-gravid uterus (FIG. 6D) resulting from administration of MTX-SS-Ps. Scale bar: 1 mm. Bottom panels: brightfield micrographs at 2.5× and 10× magnification of H&E-stained histologic serial sections, from the same fetoplacental units depicted in the top panels, were investigated at different areas of the same section to analyze specific features. em—embryo, pl—placenta, end—endometrium, myo—myometrium, peri—perimetrium.

FIGS. 7A-7C illustrate the safety evaluation of MTX polymersomes to the mother and fetus, and subsequent pregnancy. Changes in body weight of pups at post-natal day (pnd) 1 through pnd21 from 1 st pregnancy from each treatment group: control (saline, n=3), free MTX (1 mg kg-1 2×, n=3), and MTX-SS-Ps (1 mg kg-1 2×, n=3) (FIG. 7A), and subsequent 2nd pregnancy (without any treatment) (FIG. 7B). Changes in body weight of pups from treatment groups with various MTX doses: 2 mg kg-1 2×, 4 mg kg-1 2×, and 6 mg kg-1 2× (FIG. 7C).

FIGS. 8A-8C compare blood levels to evaluate cardiac, kidney, and liver function of dams following treatment with MTX-SS-Ps, free MTX, and saline (control): FIG. 8A compares total protein, albumin, and globulin; FIG. 8B illustrates cardiac function (CK) and liver function (ALT, ALP and AST); and FIG. 8C illustrates kidney function (BUN and creatinine).

FIGS. 9A and 9B present the ex vivo evaluation of murine pregnancy progress following i.v. administration at Gd6.5. FIG. 9A compares ultrasound (US) images of embryos from each treatment group: control (saline), MTX-SS-Ps (at 3 and 4 mg/kg of MTX), and free MTX (12 mg/kg MTX) at Gd6.5. Scale bar=1 mm. FIG. 9B compares photographs of resected uteri of mice (n=3) from FIG. 9A, euthanized at Gd15.5.

FIGS. 10A and 10B present the ex vivo evaluation of murine pregnancy progress following i.v. administration at Gd8.5. FIG. 10A compares ultrasound (US) images of embryos from each treatment group: control (saline), MTX-SS-Ps (at 3 and 4 mg/kg of MTX), and free MTX (12 mg/kg MTX) at Gd8.5. Scale bar=1 mm. FIG. 10B compares photographs of resected uteri of mice (n=3) from FIG. 10A, euthanized at Gd17.5.

FIGS. 11A and 11B are schematic illustrations computational ENT-1 docking analysis of 6-chloro-guanosine (Gn) ligand (FIG. 11A) within the ENT-1 protein binding cavity and (FIG. 11B) interacting with molecules of ENT-1 protein. Guanosine makes four (4) hydrogen bonds with Gln159, Trp 30, Arg314, and Asp310 residues within the binding cavity of ENT-1.

FIGS. 12A and 12B show a schematic illustration of a representative methotrexate-encapsulated Gn-targeted (Gn-MTX@SS-Ps) polymersome (FIG. 12A) and a TEM of the representative methotrexate-encapsulated Gn-targeted polymersome (FIG. 12B).

FIGS. 13A-13D illustrate the biodistribution of NIR dye-loaded non-targeted and targeted NIR@SS-Ps. Fluorescence images of mice bearing JEG-3 subcutaneous tumors (FIG. 13A) and resected major organs/tumors (FIG. 13B) mice (n=3), 12 hours post-injection (i.v.) of saline (control), NIR@SS-Ps (non-targeted, 0.3 mg/ml dye, 100 μL) and Gn-NIR@SS-Ps (targeted, 0.3 mg/mL dye, 100 μL). Semiquantitative analysis of fluorescence signal in tumors and organs (FIGS. 13C and 13D), 12 hours post-injection with NIR@SS-Ps, Gn-NIR@SS-Ps, and saline (control). Values are expressed as mean±SD, n=3. The data in FIG. 13C was assessed with one-way ANOVA, and the data in FIG. 13D was assessed with two-way ANOVA *p<0.05, **p<0.01, ***p<0.001, ****p<0.0001.

FIGS. 14A-14D illustrate ENT-1 targeted MTX-based treatment of CC. FIG. 14A is a schematic timeline of targeted anticancer therapy performed in mice 6 days following subcutaneous tumor inoculation. Free MTX, MTX@SS-Ps, Gn-MTx@SS-Ps, and saline as control were injected every second day (10 mg/kg, 6 doses total, i.v.), followed by tumor growth monitoring for 6 days (n=3). Photographs (FIG. 14B) and quantitative analysis of weights (FIG. 14C) and volume (FIG. 14D) of JEG-3 subcutaneous tumors resected from mice at day 23 after treatment with 6 doses of free MTX, MTX@SS-Ps, Gn-MTX@SS-Ps, and saline control. The data in FIGS. 14C and 14D is expressed as mean±SD, n=3, assessed with one-way ANOVA, *p<0.05, **p<0.01, ***p<0.001, ****p<0.0001.

FIGS. 15A-15D illustrate the safety evaluation of ENT-1 targeted MTX-loaded polymersomes. The body weights (FIG. 15A) and blood levels of total protein, albumin, and globulin (FIG. 15B), BUN and creatinine, illustrating kidney function (FIG. 15C), and CK, illustrating cardiac function, and ALT, ALP and AST, illustrating liver function (FIG. 15D) of mice following treatment with targeted Gn-MTX@SS-Ps vs. non-treated mice (saline control). The data in FIGS. 15B and 15C is expressed as mean±SD, n=3, assessed with two-way ANOVA.

### DETAILED DESCRIPTION

In a first aspect, the present disclosure provides methotrexate-loaded polymersomes and their use for treating ectopic pregnancy.

In this aspect, the disclosure provides a method for treating ectopic pregnancy (EP) in a subject (e.g., human subject). In certain embodiments, the disclosure provides a method for treating an ectopic pregnancy, comprising administering a therapeutically effective amount of a methotrexate-containing polymersome to a subject in need thereof. The methotrexate-containing polymersome useful in the method comprises methotrexate, or a pharmaceutically acceptable salt thereof, and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block. By virtue of the amphiphilic block copolymer, the polymersome has a bilayer structure with a hydrophilic interior core, hydrophobic layer, and a hydrophilic outer shell formed by the hydrophilic PEG block (i.e., the polymersome has a bilayer formed from the hydrophobic PCL block, a hydrophilic interior core and a hydrophilic outer shell formed by the hydrophilic PEG block). Methotrexate, or a pharmaceutically acceptable salt thereof, is encapsulated in the hydrophilic interior core.

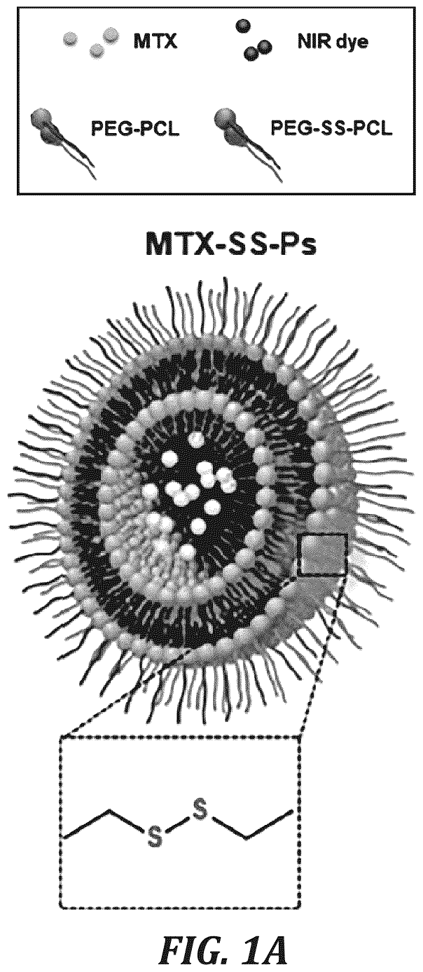

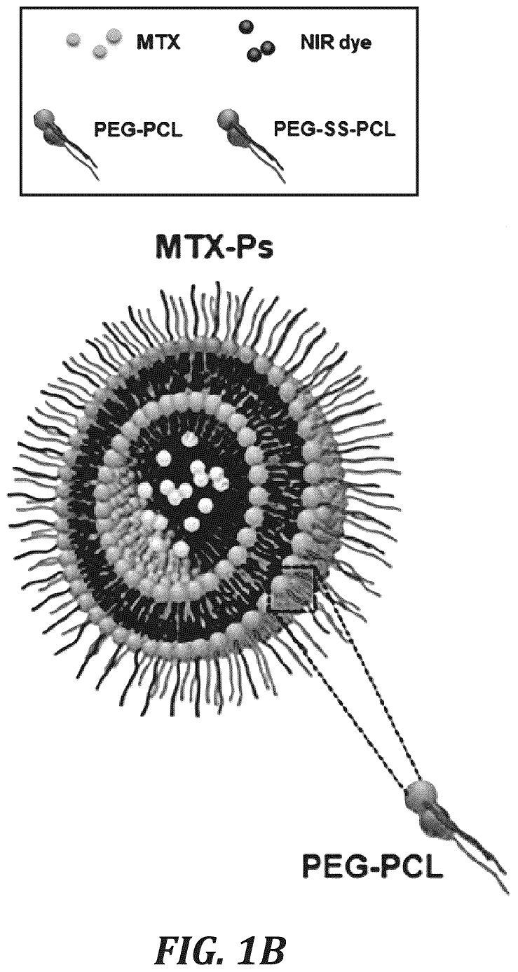

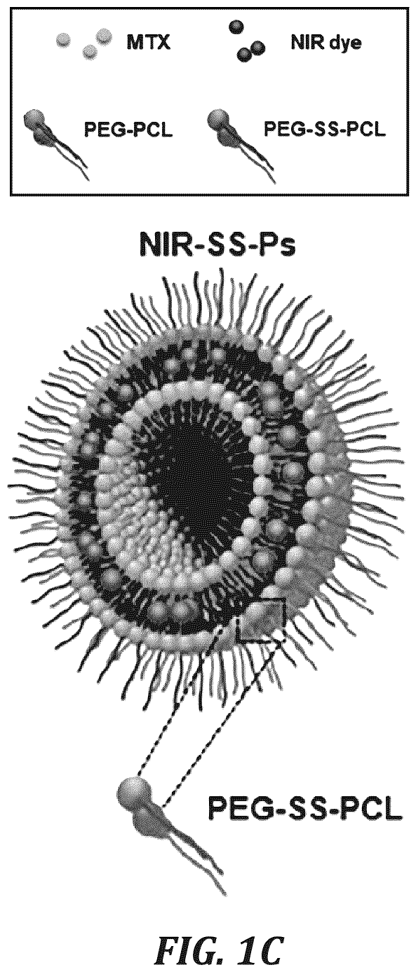

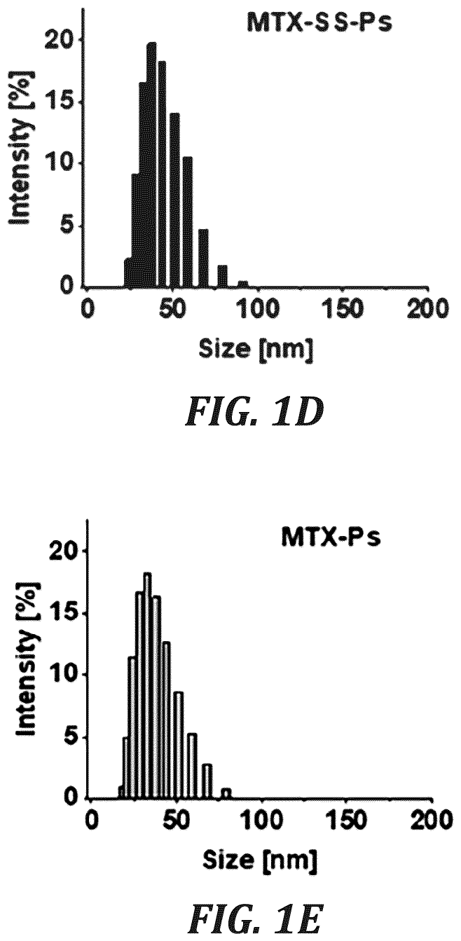

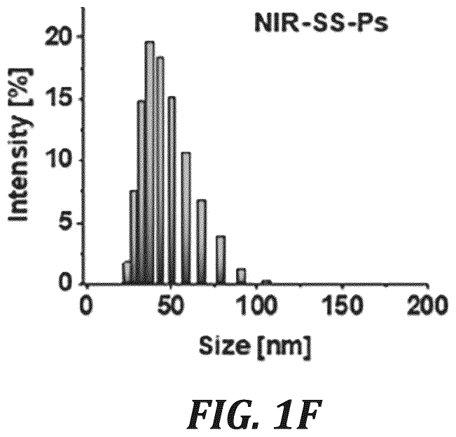

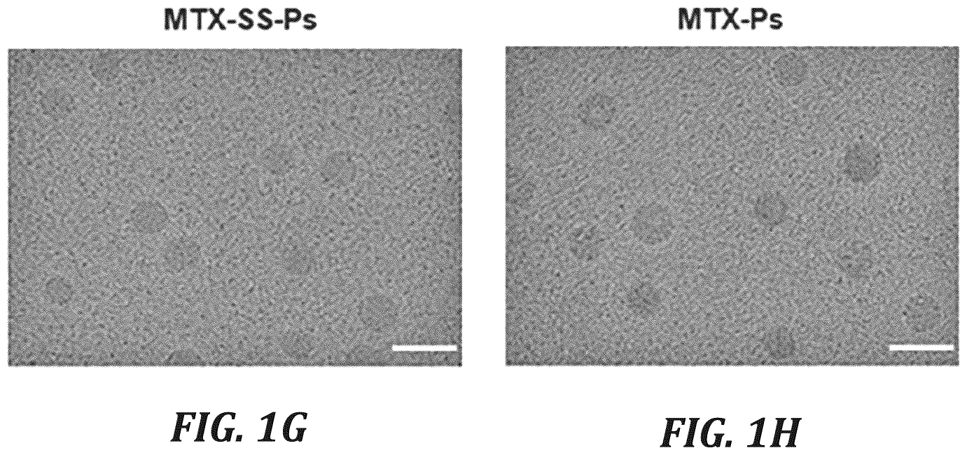

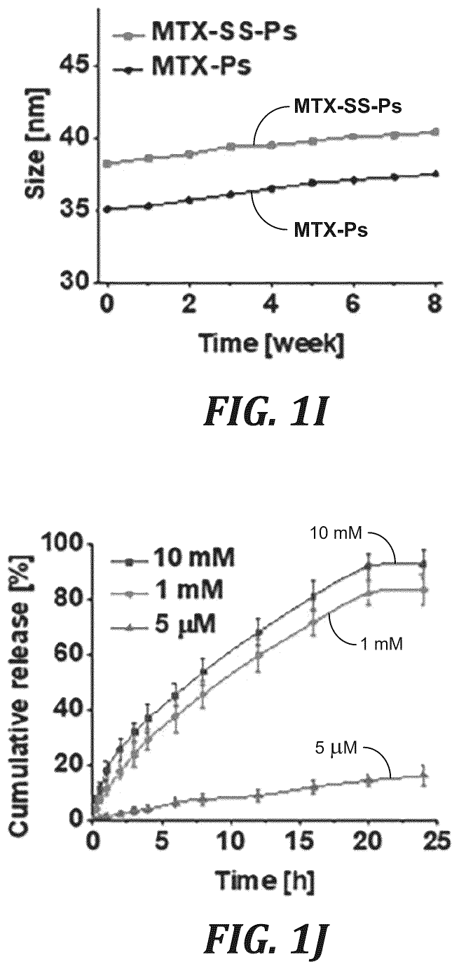

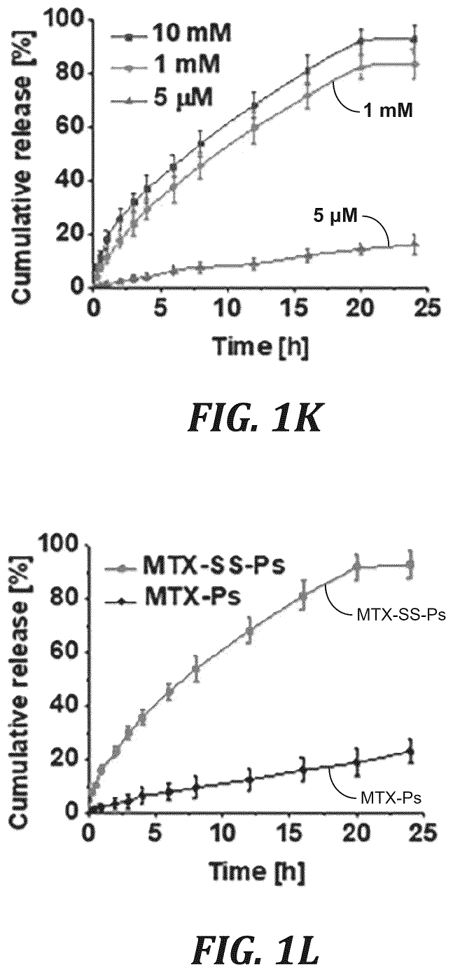

As used herein, the term “polymersome” refers to a self-assembled bilayer polymeric vesicle composed of an amphiphilic diblock copolymer (e.g., block polyethylene glycol-disulfide-polycaprolactone (PEG-SS-PCL) polymer) having the capability of encapsulating water-soluble molecules such as methotrexate (MTX) (e.g., sodium salt) within the hydrophilic core and hydrophobic molecules (e.g., NIR dye) within the hydrophobic bilayer. FIGS. 1A-1C depict representative polymersomes of the disclosure. In certain embodiments, the methotrexate-containing polymersome of the disclosure are prepared using a microfluidic mixing approach.

The methotrexate-containing polymersome disclosed herein is formed from an amphiphilic PEG-PCL copolymer containing a stimuli-sensitive disulfide bond between the PEG and PCL blocks (PEG-SS-PCL; see, for example, formula II below for PEG(2k)-SS-PCL(5k)) for glutathione (GSH)-triggered intracellular release of the drug cargo. The high concentrations of intracellular GSH found in placental cells rapidly reduce the incorporated disulfide bond within the polymersome upon internalization into placental cells resulting in the disintegration of the polymersome and efficient drug (MTX) release.

The methotrexate-containing polymersome disclosed herein is distinguished from other nanoparticulate forms, such as nanocrystals formed by precipitation methods, prepared from the same or similar components and that do not have the advantageous structural characteristics of the polymersome described herein. The methotrexate-containing polymersome disclosed herein are not nanocrystals having amorphous or crystalline forms. The advantageous structure and properties of the methotrexate-containing polymersome disclosed herein is due in part to the method for preparing the polymersome. In certain embodiments, the methotrexate-containing polymersome disclosed herein are prepared by microfluidic mixing of a first solution (water-miscible organic, such as acetone) of the amphiphilic block polyethylene glycol-disulfide-polycaprolactone polymer and a second solution (aqueous) of methotrexate (or water-soluble salt thereof), as described in the Experimental section below.

In certain embodiments of the method, ectopic pregnancy is successfully treated by administration of a dose of the methotrexate-containing polymersome. As used herein, the term “successfully treated” refers to the resolution of hCG to non-pregnant levels (e.g., safe termination of ectopic pregnancy, which is a non-viable pregnancy). In certain of these embodiments, the methotrexate-containing polymersome contains from about 20 to about 50 mg methotrexate per 10 mg polymersome. In other embodiments, the methotrexate-containing polymersome contains from about 20 to about 50 mg methotrexate, from about 25 to about 45 mg methotrexate, from about 30 to about 40 mg methotrexate, or from about 35 mg methotrexate per from about 9 to about 15 mg polymersome, from about 10 to about 14 mg polymersome, from about 11 to about 13 mg polymersome, or about 12 mg polymersome, respectively. In one embodiment, the methotrexate-containing polymersome contains from about 35 mg methotrexate per 12 mg polymersome (i.e., 3:1 w/w methotrexate:polymersome).

In certain embodiments of the method, ectopic pregnancy is successfully treated by administration of a single dose of the methotrexate-containing polymersome.

In other embodiments, ectopic pregnancy is successfully treated by administration of two or more doses of the methotrexate-containing polymersome. In certain of these embodiments, the two doses are systemically administered from about 1 to about 4 days apart.

In certain embodiments, therapeutically effective amounts of methotrexate (or equivalent pharmaceutically acceptable salt thereof) administrated by the methods range from about 1 mg/m² to about 50 mg/m² (methotrexate/subject surface area). In other embodiments, the amount of methotrexate (or equivalent pharmaceutically acceptable salt thereof) administrated by the methods range from about 10 mg/m² to about 50 mg/m², from about 10 mg/m² to about 25 mg/m², from about 10 mg/m² to about 20 mg/m², from about 10 mg/m² to about 15 mg/m² (methotrexate/subject surface area). In one embodiment, the amount of methotrexate (or equivalent pharmaceutically acceptable salt thereof) administrated by the method is about 12.5 mg/m² (methotrexate/subject surface area).

In certain embodiments, therapeutically effective amounts of methotrexate (or equivalent pharmaceutically acceptable salt thereof) administrated by the methods range from about 0.1 mg/kg to about 2.0 mg/kg (methotrexate/subject body weight). In certain of these embodiments, therapeutically effective amounts of methotrexate administrated by the methods range from about 0.2 to about 1.9, about 0.3 to about 1.8, about 0.4 to about 1.7, about 0.5 to about 1.6, about 0.6 to about 1.5, about 0.7 to about 1.4, about 0.8 to about 1.3, about 0.9 to about 1.2, or about 0.9 to about 1.1 mg/kg (methotrexate/subject body weight). In one embodiment the therapeutically effective amount of methotrexate (or equivalent pharmaceutically acceptable salt thereof) administrated by the methods is about 1.1 mg/kg (methotrexate/subject body weight).

In the methods described herein, the methotrexate-containing polymersome is systemically administered by, for example, intravenous injection. For such administration, the polymersomes described herein are formulated as pharmaceutical compositions with a pharmaceutically acceptable carrier or diluent. Suitable carriers include solutions for injection, such as saline and dextrose solutions.

In certain embodiments, the methotrexate-containing polymersome described herein is in lyophilized form that may be reconstituted at the site for administration (e.g., patient's bedside) with an injectable carrier to provide a solution for administration.

In another aspect of the disclosure, methotrexate-containing polymersomes are provided. In certain embodiments, the methotrexate-containing polymersome comprises:
(a) methotrexate, or a pharmaceutically acceptable salt thereof, and
(b) an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block, and
wherein methotrexate, or a pharmaceutically acceptable salt thereof, is encapsulated in the hydrophilic interior core.

In certain embodiments, the hydrophilic PEG block has a molecule weight from about 2 kDa to about 10 kDa. In certain of these embodiments, the hydrophilic PEG block has a molecule weight of about 2 kDa.

In certain embodiments, the hydrophobic PCL block has a molecule weight from about 5 kDa to about 10 kDa. In certain of these embodiments, the hydrophobic PCL block has a molecule weight of about 5 kDa.

In one embodiment, the hydrophilic PEG block has a molecule weight of about 2 kDa and the hydrophobic PCL block has a molecule weight of about 5 kDa.

In certain embodiments, the methotrexate-containing polymersome contains from about 20 to about 50 mg methotrexate per 10 mg polymersome. In other embodiments, the methotrexate-containing polymersome contains from about 20 to about 50 mg methotrexate, from about 25 to about 45 mg methotrexate, from about 30 to about 40 mg methotrexate, or from about 35 mg methotrexate per from about 9 to about 15 mg polymersome, from about 10 to about 14 mg polymersome, from about 11 to about 13 mg polymersome, or about 12 mg polymersome, respectively. In one embodiment, the methotrexate-containing polymersome includes from about 35 mg methotrexate per 12 mg polymersome (i.e., 3:1 w/w methotrexate:polymersome).

As noted above, for certain embodiments of the dosage forms, methotrexate is present in the polymersome in an amount of about 0.75 mg per 1.0 mg polymersome.

In certain embodiments, the polymersome described herein has a hydrodynamic size from about 25 to about 90 nm. In other embodiments, the polymersome described herein has a hydrodynamic size from about 30 to about 75 nm, from about 35 to about 50 nm, or from about 38 to about 42 nm. In one embodiment, the polymersome described herein has a hydrodynamic size of about 38 nm.

In certain embodiments, the polymersome described herein has a polydispersity index about 0.01 to about 0.2. In other embodiments, the polymersome described herein has a polydispersity index from about 0.05 to about 0.15, or from about 0.06 to about 0.12. In one embodiment, the polymersome described herein has a polydispersity index of about 0.11 nm.

In a further aspect, the disclosure provides a method for making the methotrexate-containing polymersome described herein. In certain embodiments, the method comprises microfluidic mixing of a first solution of an amphiphilic block polyethylene glycol-disulfide-polycaprolactone polymer (water-miscible organic solvent, e.g., methanol, ethanol, acetone) and a second solution of methotrexate, or pharmaceutically acceptable salt thereof (aqueous solution), wherein the first and second solutions are water miscible.

In a related aspect, the disclosure provides an imaging agent-containing polymersome. The imaging agent-containing polymersome is a diagnostic agent companion to the therapeutic methotrexate-containing polymersome. The companion diagnostic polymersome is prepared from the same components as for the methotrexate-containing polymersomes described herein except that the diagnostic agent does not include methotrexate and does include an imaging agent. The imaging agent-containing polymersome is a companion diagnostic agent that can be used for imaging ectopic pregnancy in conjunction with the therapeutic methotrexate-containing polymersome described herein. The imaging agent-containing polymersome includes an imaging agent (e.g., a hydrophobic, photostable, near infra-red dye (silicon 2,3-naphthalocyanine bis(trihexylsilyloxide) (SiNc) encapsulated in the polymersome lipid bilayer. See, for example, FIG. 1C. Other suitable imaging agents useful in the imaging agent-containing polymersome include fluorescence and photoacoustic imaging agents. Depending on the nature of the imaging agent, the imaging agent is either encapsulated in the polymersome core (i.e., hydrophilic imaging agent) or the polymersome lipid bilayer (i.e., hydrophobic imaging agent).

The following description is directed to representative methotrexate-containing polymersomes and their use in treating ectopic pregnancy.

Because systemic MTX treatment is associated with substantial side effects, enhancing its precise delivery at an effective dose to the site of ectopic implantation and, consequently, increasing its safety and efficacy, can greatly improve therapeutic results.

This disclosure provides the first glutathione-responsive polymersomes for efficient delivery of MTX to the implantation site and its triggered release in placental cells. Fluorescence and photoacoustic imaging have confirmed that the developed polymersomes preferentially accumulate after systemic administration in the implantation site of pregnant mice at early gestational stages. The high concentrations of intracellular glutathione reduce an incorporated disulfide bond within polymersomes upon internalization into placental cells, resulting in their disintegration and efficient drug release. Consequently, MTX delivered by polymersomes induces pregnancy demise in mice, as opposed to free MTX at the same dose regimen. To achieve the same therapeutic efficacy with free MTX, a 6-fold increase in dosage is required. In addition, mice successfully conceive and birth healthy pups following a prior complete pregnancy demise induced by methotrexate polymersomes. Therefore, the developed MTX nanomedicine can potentially improve EP management and reduce associated mortality rates and related cost.

This disclosure relates to nanomedicines for EP management based on specifically designed nanoparticles with effective drug delivery and release at the implantation site, aiming at decreasing the necessary dose and adverse effects of MTX while enhancing its therapeutic effect. The disclosure provides a nanomedicine strategy to improve EP management, demonstrating proof-of-concept in a pregnant mouse model. The disclosure provides glutathione-responsive MTX polymersomes comprised of an amphiphilic polyethylene glycol-disulfide-polycaprolactone (PEG-SS-PCL) copolymer, containing a disulfide bond between PEG and PCL blocks for efficient delivery and triggered intracellular release of the drug cargo in the placenta (FIG. 1A).

### Synthesis and Characterization of MTX-Loaded and Companion Imaging Polymersomes

Because MTX remains the primary treatment for EP, it was hypothesized that MTX would function more effectively and safely when encapsulated within a specifically constructed biocompatible nanocarrier capable of effective drug delivery and release at the implantation site. Polymersomes, self-assembled bilayer polymeric vesicles that are composed of amphiphilic di-block copolymers, were selected for this purpose due to their capability of encapsulating water-soluble molecules such as MTX (sodium salt) within the hydrophilic core and hydrophobic molecules (e.g., NIR dye) within the hydrophobic bilayer (see FIGS. 1A-1C). Using a microfluidic mixing approach, MTX-loaded polymersomes were prepared composed of an amphiphilic di-block copolymer with a hydrophobic polycaprolactone (PCL) block to generate the stable polymersome bilayer and a polyethylene glycol (PEG) block to create a hydrophilic core as well as a water-soluble shell. In addition, the polymersomes were also constructed from an amphiphilic PEG-PCL copolymer containing a disulfide bond between the PEG and PCL blocks (PEG-SS-PCL) for glutathione (GSH)-triggered intracellular release of the drug cargo.

Representative amphiphilic PEG-PCL block copolymers useful for preparing the polymersomes described herein are shown below. A representative amphiphilic PEG-PCL block copolymer without a disulfide bond intermediate the PEG and PCL blocks (i.e., methoxy poly(ethylene glycol)-b-poly(ε-caprolactone)) (e.g., PEG(2k)-PCL(5k)) has formula I:

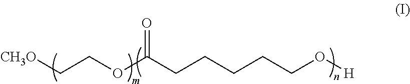

A representative amphiphilic PEG-PCL block copolymer with a disulfide bond intermediate the PEG and PCL blocks (i.e., methoxy poly(ethylene glycol)-b-disulfide-poly(ε-caprolactone)) (e.g., PEG(2k)-SS-PCL(5k)) has the formula II:

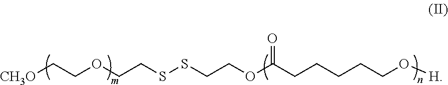

For the representative block copolymers shown above, PEG(2k)-PCL(5k) and PEG(2k)-SS-PCL(5k), m=—(CH₂CH₂O)— repeating units sufficient to provide a molecular weight of 2 kDa for the PEG block and n=—(C(═O)(CH₂)₅O)— repeating units sufficient to provide a molecular weight of 5 kDa for the PCL block.

The high concentrations of intracellular GSH are expected to rapidly reduce an incorporated disulfide bond within the polymersome upon internalization into placental cells, resulting in the disintegration of nanocarriers and efficient drug release. The developed disulfide bond-containing polymersomes loaded with MTX (MTX-SS-Ps, FIG. 1A) have a spherical shape (FIG. 1G), nearly neutral surface charge (−2.62±0.45 mV) (Table 1), uniform distribution (PDI (polydispersity index) of 0.11±0.02) and a hydrodynamic size of 38.2±0.4 nm (FIG. 1D), that is comparable in size and charge to the same polymersome lacking a disulfide bond (MTX-Ps, FIGS. 1E and 1H), or when loaded with NIR dye (FIG. 1F and Table 1). Storage of MTX-loaded polymersomes for 8 weeks at 4° C. resulted in only a minimal change in size (FIG. 1I), demonstrating favorable stability of this formulation and extended shelf life.

TABLE 1
Nanoparticle size (by DLS, diameter in nm ±
standard deviation (SD)), polydispersity index
(PDI) ± SD, and zeta potential (mV ± SD).

| Nanoagent | Size (nm) | PDI | Zeta potential (mV) |
|---|---|---|---|
| MTX-SS-Ps | 38.2 ± 0.4 | 0.11 ± 0.02 | −2.62 ± 0.45 |
| MTX-Ps | 35.5 ± 0.3 | 0.10 ± 0.01 | −2.35 ± 0.29 |
| NIR-Ps | 37.4 ± 0.2 | 0.11 ± 0.03 | −0.29 ± 0.14 |
| NIR-SS-Ps | 39.3 ± 0.5 | 0.12 ± 0.02 | −0.58 ± 0.12 |

The release of MTX from polymersomes (MTX-Ps and MTX-SS-Ps) was evaluated in the presence and absence of GSH at physiological pH (7.4) (FIGS. 1J-1L). Cytosolic concentrations of GSH are between 1 to 10 mM, while plasma concentrations of GSH are within 1 to 5 μM. As shown in FIG. 1J, MTX-SS-Ps efficiently release the drug at both 1 and 10 mM GSH concentrations, exceeding 90% release at 10 mM GSH and 80% release at 1 mM GSH, reaching a plateau at about 20 hours. However, at 5 μM GSH, only a negligible release of drug from MTX-SS-Ps was observed after 7 hours, and less than 15% release after 20 hours. MTX release from both MTX-Ps and MTX-SS-Ps in PBS at pH 7.4 follows a similar pattern in the absence of GSH with a minimal release (FIG. 1K). When comparing the MTX release from MTX-Ps to that of MTX-SS-Ps at 10 mM GSH (FIG. 1L), drug release from MTX-Ps was minimal and resembled the release pattern observed for MTX-Ps in the absence of GSH. These release studies indicates that MTX-SS-Ps ensures the GSH-triggered intracellular release of MTX for effective therapeutic outcomes.

Naphthalocyanine derivatives, when encapsulated within polymeric nanoparticles, are capable of producing a strong photoacoustic (PA) signature that is distinct from the background. As described herein, a polymersome-based companion imaging agent was prepared by encapsulating silicon 2,3-naphthalocyanine bis(trihexylsilyloxide) (SiNc), a hydrophobic, photostable, near infra-red dye (FIG. 1C). Polymersomes loaded with SiNc (NIR-SS-Ps) demonstrate a strong fluorescence signal in aqueous solution at about 780 nm, as well as a strong and narrow PA peak at about 780 nm, a region of the electromagnetic spectrum where endogenous contrast agents, such as oxygenated and deoxygenated hemoglobin, demonstrate their lowest PA signal. The aforementioned PA properties, confirmed in solution studies, make these candidates for in vivo imaging.

### Biodistribution of Developed Polymersomes in Pregnant Mice

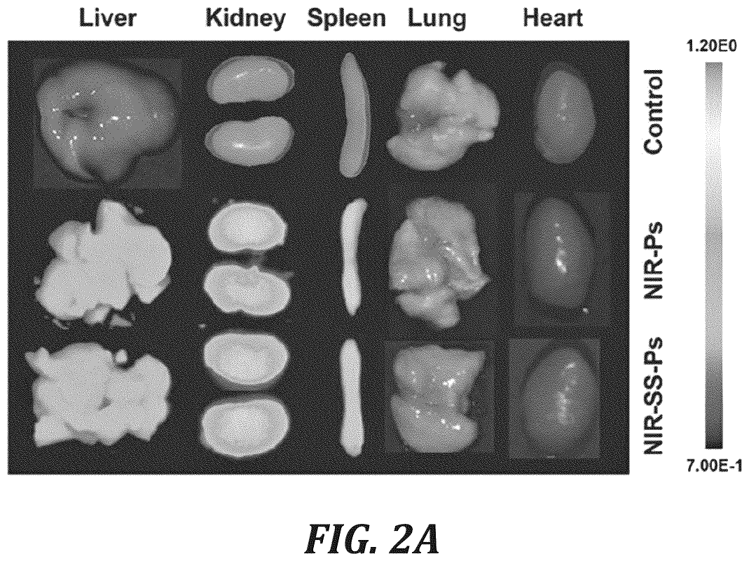

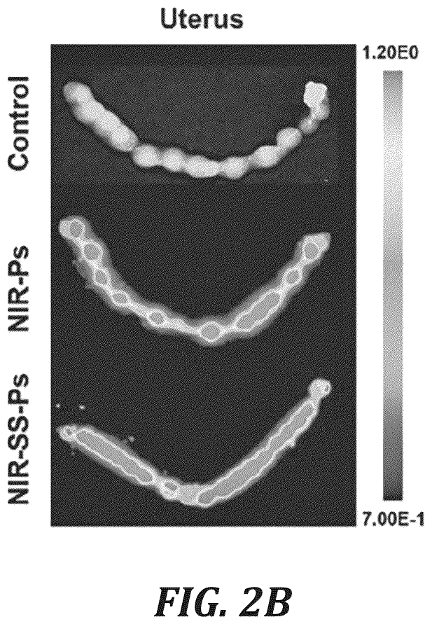

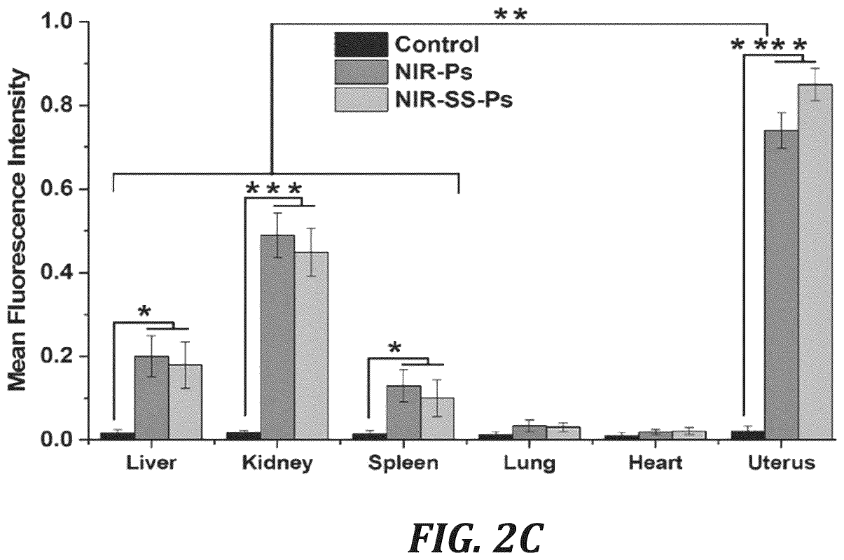

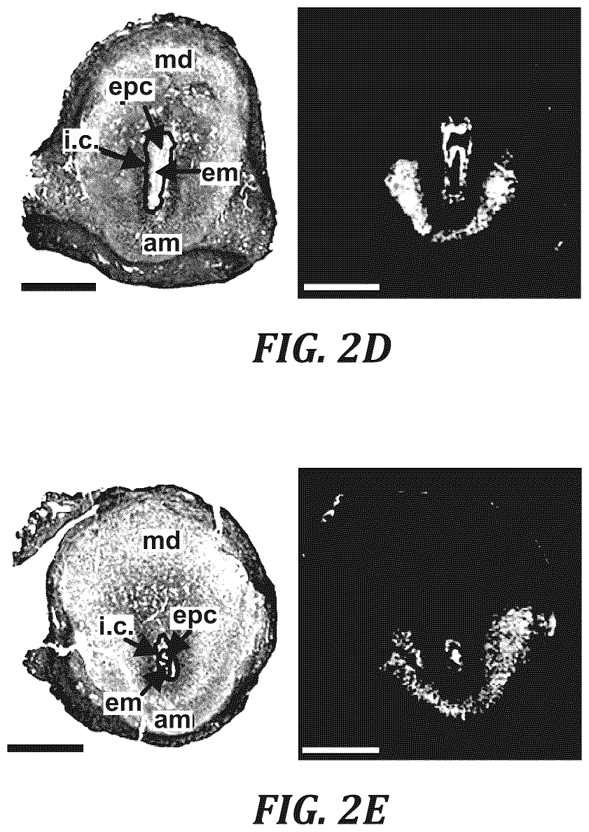

In the absence of an animal model for EP, the delivery of imaging and therapeutic agents to the placenta has been studied using the pregnant murine model since the human and mouse placenta are anatomically and functionally comparable. Additionally, while the location of implantation varies between normal and ectopic pregnancies, the mechanisms of implantation and placentation remain the same. Thus, with the goal of timely management of EP, the distribution of NIR dye-loaded polymersomes were evaluated with and without disulfide bonds (NIR-SS-Ps and NIR-Ps) in a pregnant mouse model at gestational day (Gd) 6.5, which corresponds to the early phase of the first trimester of a human pregnancy (FIGS. 2A-2E). Because the process of implantation and placentation is the same regardless of location, a goal was to demonstrate that MTX-loaded polymersome with S—S-bond can accumulate in the developing vascularized placenta, efficiently release MTX in placental cells with high levels of GSH, and as a result cause the demise of the associated fetus. The implication would be that EP, presumably with a similarly vascularized placenta, could act as a sink for the polymersome following systemic administration, and the released MTX would provide the required therapeutic effect.

At 24 hours post-intravenous (i.v.) injection, both NIR-Ps and NIR-SS-Ps produce a prominent fluorescence signal in each implantation site of the gravid uterus (FIG. 2B). Fluorescence imaging of resected organs revealed similar accumulation of both NIR-Ps and NIR-SS-Ps in uteri and major organs of pregnant mice at Gd7.5—mostly in kidney, liver, and spleen—24 hours following i.v. administration at Gd6.5 (FIGS. 2A and 2C). While some accumulation of nanoparticles was observed in the lungs and heart, the signal was similar to background levels, and there was no statistically significant difference in fluorescence intensity between these organs and those of control mice injected with saline. The results show that the polymersomes preferentially accumulate in the uterus as compared to other organs. For instance, the fluorescence signal of NIR polymersomes in kidneys and liver was about 50% and 80% lower than that observed in the uterus, respectively (FIG. 2C).

The mean fluorescence signal generated by NIR-SS-Ps (0.18±0.06) in the liver is only 10% lower than the signal (0.20±0.05) of the non-responsive polymersome (NIR-Ps), and this difference is not statistically significant (FIG. 2C). Similarly, the mean fluorescence signal generated by NIR-SS-Ps (0.45±0.06) in the kidneys is only 8% lower than the signal (0.49±0.05) of the non-responsive polymersome (NIR-Ps), and this difference is also not statistically significant. Therefore, NIR-SS-Ps and NIR-Ps demonstrate similar accumulation in various tissues following systemic administration. The similar biodistribution of both formulations is expected because NIR-SS-Ps and NIR-Ps have very similar hydrodynamic size (39.3 nm vs 37.4 nm, Table 1), charge (−0.58 mV vs −0.29 mV) and surface modification (2 kDa PEG vs 5 kDa PEG).

Following ex vivo assessment of polymersome distribution in organs and implantation sites 24 hours after administration with a small animal imaging system, fluorescence microscopy was employed to further characterize the distribution of polymersomes in fetoplacental tissues of mice administered NIR-Ps and NIR-SS-Ps (FIGS. 2D and 2E). The intense fluorescence signal was observed in the antimesometrial decidua, the ectoplacental cone, and the region surrounding the implantation crypt, as confirmed by subsequent H&E staining of the same tissue sections (FIGS. 2D and 2E). No fluorescence signal was observed within the primitive streak-stage embryo.

In addition to the biodistribution profile of NIR-SS-Ps at 24 hours after injection, their distribution in the uterus and other organs at earlier time points was also examined. The results revealed that the developed polymersomes efficiently accumulate in the implantation sites as early as 1 hour after injection. At this time point, NIR fluorescence generated by NIR-SS-Ps was also detected in the liver, kidneys, lungs, heart, and spleen, albeit at a lower intensity than at the implantation sites. The findings further revealed that polymersomes were significantly eliminated from the aforementioned organs within 12 hours while remaining at the implantation sites.

The disulfide bond containing polymersomes with a hydrodynamic size of about 39 nm, nearly neutral surface charge and a 2 kDa PEG coating were developed. The biodistribution studies demonstrated that polymersomes with the above-mentioned physicochemical parameters can efficiently accumulate and retain in the placenta while being substantially eliminated from other organs. The mechanism of nanoparticle retention in the placenta when compared to other organs remains unclear.

In Vivo Photoacoustic Visualization of Murine Placenta with the Developed Polymersomes

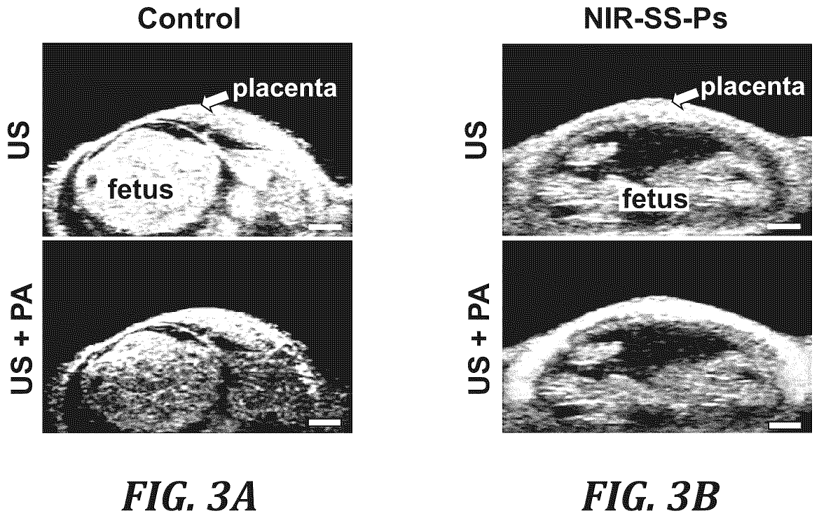

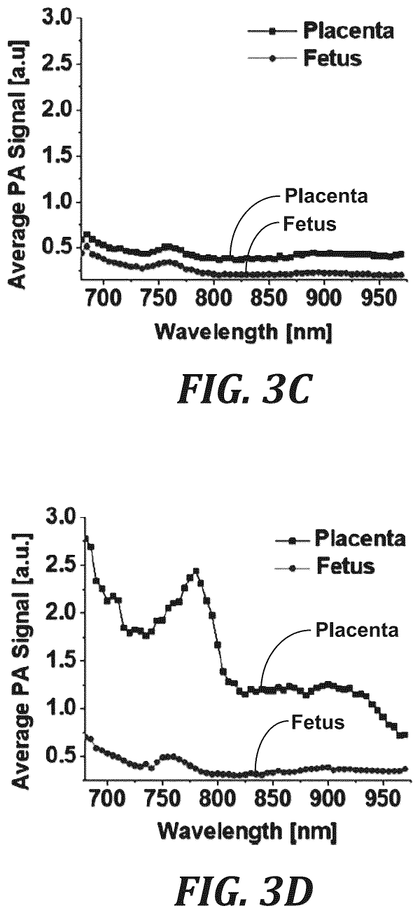

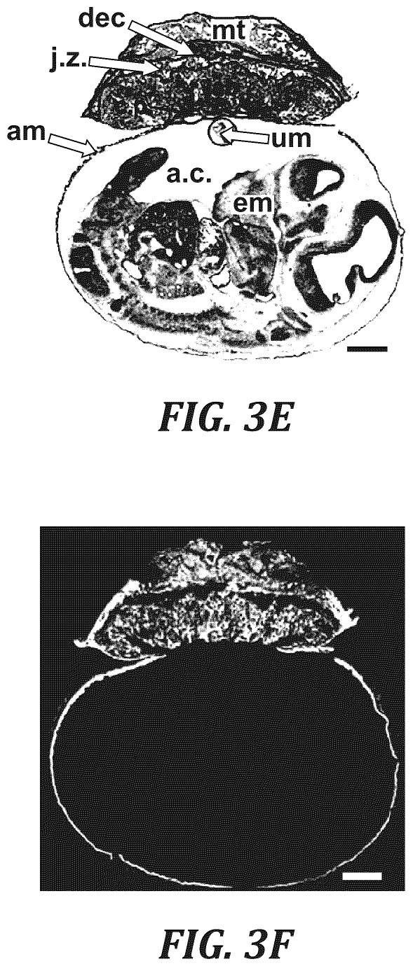

As the detection of extrauterine gestation is the most accurate indicator of EP, advancements in real-time imaging technologies are greatly valuable for EP diagnosis. Photoacoustic imaging (PAI) offers deeper tissue penetration than other optical modalities, such as fluorescence imaging, due to its generation of acoustic waves, which encounter significantly less scattering and subsequent signal attenuation than optical waves. PAI operates without the use of harmful ionizing radiation and exploits the natural endogenous photoacoustic contrast of tissue components, such as melanin and blood. Exogenous contrast agents with pronounced PA signals further enhance clear distinction of targeted tissue from the background (tissue, blood, or water). Therefore, the PAI capabilities of NIR polymersomes as well as their distribution in the murine placenta following the establishment of blood flow was evaluated. In humans, the main placental structure forms by day 21 and maternal blood flow is established by day 80, and both of these processes are completed in the first trimester of human pregnancy, which corresponds to early gestation in mice of Gd6.5-13.5. Thus, the effectiveness of NIR polymersome as a PA contrast agent was evaluated for visualizing the fetoplacental unit at Gd12.5. It was believed that NIR-SS-Ps would concentrate in the rapidly dividing cells and highly vascular tissues of the growing placenta after systemic administration. The placental labyrinth contains an extensive network of blood flow and enables the transfer of oxygen and nutrients between the maternal and fetal circulations, suggesting that NIR polymersomes would localize preferentially within the labyrinth. Ultrasound (US) and PA images were acquired with the same transducer enabling overlay of anatomical characteristics of US images with the photoacoustic signal. A clear, intense PA signal was observed in the amnion and placenta 24 hours post-injection, but not in the fetus, of mice administered NIR-SS-Ps at Gd12.5 (FIG. 3B), when compared to the same tissues of control mice administered saline, which only displayed PA signal for blood (FIG. 3A). Recorded PA spectra confirm these findings, as evidenced by a spectral peak for NIR dye (780 nm) in the placenta of mice administered NIR-SS-Ps and the absence of this signal in the fetus (FIG. 3D), compared to control mice administered saline, where only spectral background signal is observed in fetus and placenta (FIG. 3C).

The histological analysis of the implantation sites further indicates that the developed nano-formulations are predominantly localized in the placenta (FIG. 3F). Fluorescence microscopy was used to confirm NIR-SS-Ps distribution in fetoplacental units of mice injected at Gd12.5. The intense fluorescence signal was observed in the placental labyrinth and decidua but absent from the junctional zone that separates these regions (FIGS. 3E and 3F). Fluorescence was also observed in the avascular amnion surrounding the fetus (embryo) but completely absent from the fetus itself. There is no indication that the nanoparticle reaches the fetus at mid-gestation, indicating that NIR polymersomes are not transported across the placental barrier. Hence, these disulfide-containing polymersome delivery vehicles can be employed to transport other imaging and therapeutic cargos, for the treatment of pregnancy complications or other diseases during pregnancy, without endangering the fetus.

Together, the data indicates that NIR polymersomes efficiently accumulate in implantations during early mouse pregnancy (Gd6.5-13.5), before and after placental blood flow is established (comparable to 10 weeks of human pregnancy) and facilitate PA visualization of the placenta.

### Evaluation of Therapeutic Efficacy of MTX Polymersomes

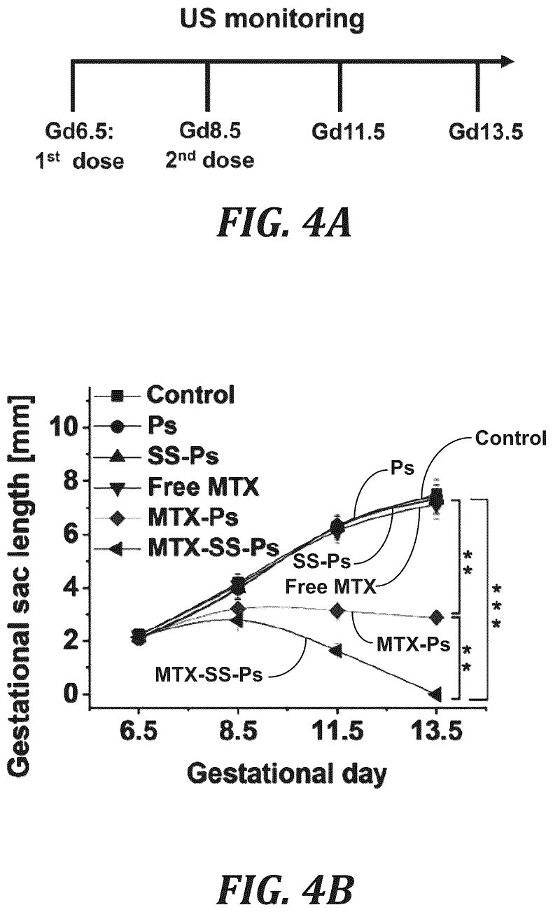

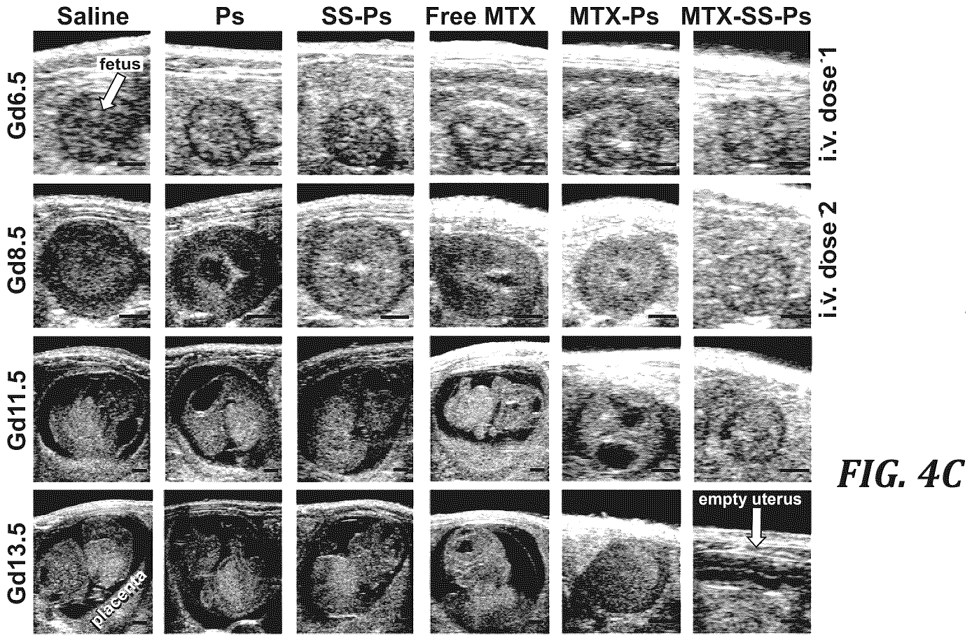

The nanomedicine approach described herein has the potential to address issues with small molecule methotrexate-chemotherapy for EP, by increasing the efficacy of delivery and release of MTX at the implantation site thanks to specifically designed nanocarriers. Embryonic development in CD-1 pregnant dams was monitored using a US imaging system (Vevo 2100, VisualSonics, Canada) every other day through Gd13.5 after i.v. administration of a two-dose regimen (FIG. 4A) of either MTX-SS-Ps (1 mg kg-1 of MTX at Gd6.5 and 8.5) or various controls (saline, free MTX (1 mg kg-1 at Gd6.5 and 8.5), empty polymersome (Ps), empty disulfide containing polymersome (SS-Ps), or MTX-Ps (1 mg kg-1 of MTX at Gd6.5 and 8.5)).

Although free MTX was administered at the clinically recommended dose for EP treatment of 1 mg kg-1, MTX did not affect fetal development in the pregnant murine model, similar to saline and empty polymersomes, according to US imaging and measurements of the gestational sac length (FIGS. 4B and 4C). The administration of MTX-Ps at the same dosing regimen (1 mg kg-1 at Gd6.5 and 8.5) demonstrated inhibition of fetal development after the 2nd dose as seen by US, indicating increased MTX delivery to the implantation site as compared to free MTX at the same dosing regimen. However, while fetal development was severely inhibited by treatment with MTX-Ps, fetal and placental tissues had not been completely resorbed by Gd13.5. In contrast, US imaging revealed that MTX-SS-Ps with a disulfide bond effectively inhibited fetal development after gestational day 8.5, and pregnancies were completely resorbed by Gd13.5 5 in all mice (n=3) treated with MTX-SS-Ps (FIG. 4C). This pronounced therapeutic effect can be attributed to improved MTX delivery to the placenta using the constructed nanocarrier (FIGS. 2A-2E) and efficient triggered MTX release from the disulfide polymersomes in the presence of high cytosolic concentrations of GSH within placental cells. The drug release studies demonstrate that about 80% of MTX is released from the S—S-bond-containing nanocarrier within 20 hours in the presence of GSH at the concentration of 1 mM and higher (FIG. 1J). GSH is present in most mammalian cells at concentrations ranging from 1 to 10 mM, with the highest levels of GSH found in the liver at a concentration of 10 mM. Previous reports also demonstrate that the level of GSH in the placenta of pregnant mice and rats is only about 1.8 times lower than in the liver. Therefore, MTX can be efficiently released from MTX-SS-Ps after accumulation in the placenta containing GSH at concentrations greater than 1 mM. The results demonstrate the impact that the SS bond, incorporated within the polymersome-based carrier, exerts on drug release and, consequently, therapeutic efficacy.

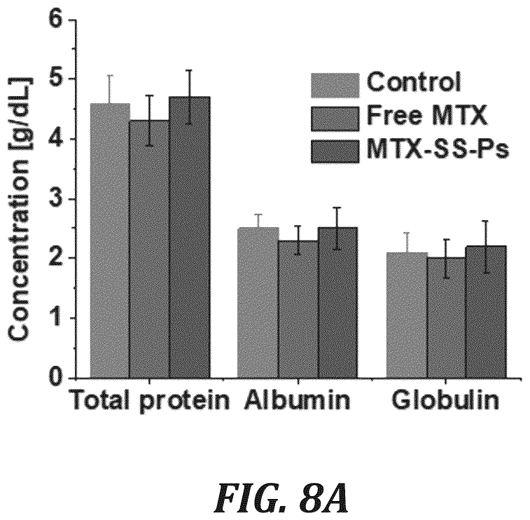

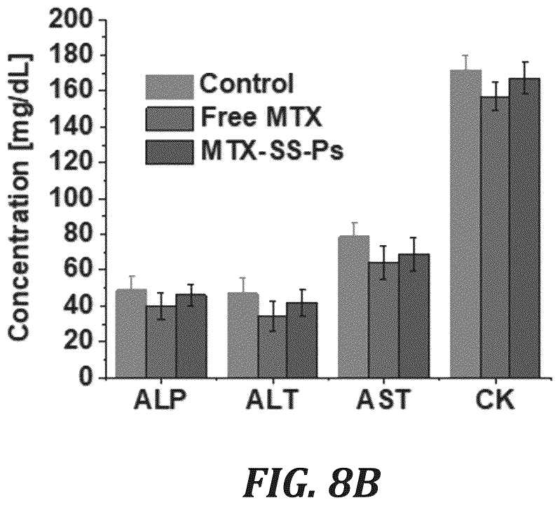

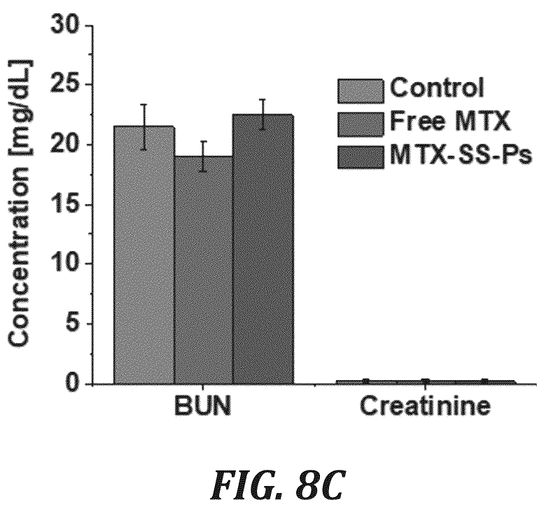

To evaluate the safety of MTX polymersomes, blood samples were collected following euthanization of treated mice at Gd13.5 and measured serum concentrations of proteins and surrogate markers indicating hepatic (alkaline phosphatase (ALP), alanine transaminase (ALT), aspartate aminotransferase (AST)), cardiac (creatine kinase (CK), and renal (blood urea nitrogen (BUN) and creatinine) function. The obtained values in the treatment (MTX-SS-Ps) and control groups were similar, with no statistically significant differences between groups, suggesting that MTX-SS-Ps are safe and well-tolerated (see FIGS. 8A-8C).

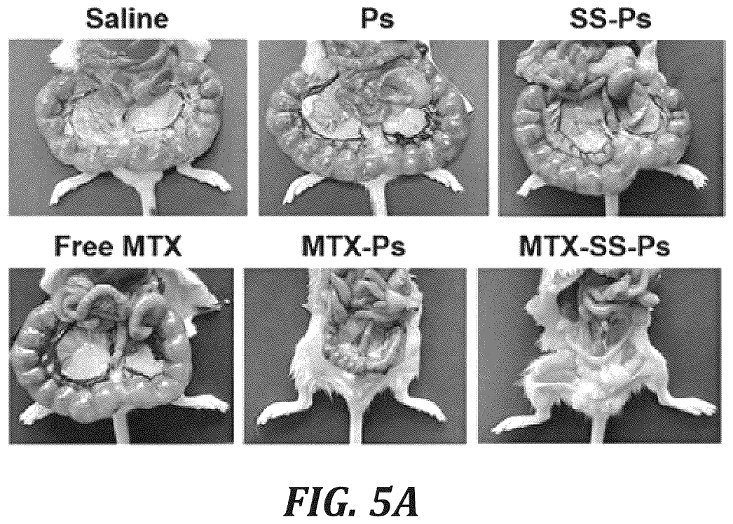

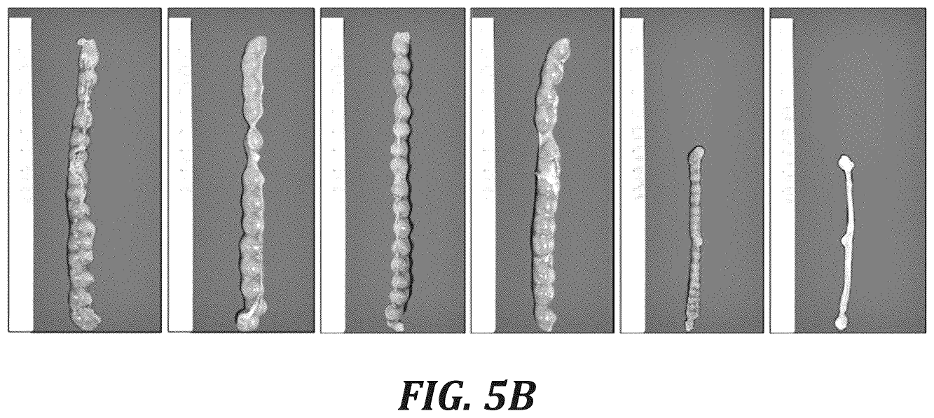

After euthanization at Gd13.5, murine uteri were exteriorized for a more detailed evaluation of fetal development (FIGS. 5A and 5B). Uteri from the saline, Ps, SS-Ps, and free MTX groups were virtually indistinguishable from one another, each containing a large litter of apparently healthy, well-developed fetuses and associated placentae (FIGS. 5A and 5B). Uteri of mice administered MTX-Ps contained severely underdeveloped embryos and no discernable placentae, and the length of the extended uterus was similar to that of a non-pregnant mouse. Distinctively, administration of MTX-SS-Ps, at the same dosing regimen of MTX given in the free MTX and MTX-Ps groups (1 mg kg-1 2×), had a profound effect on implantations, resulting in complete fetal resorption and the uteri resembling those of non-pregnant mice (FIGS. 5A and 5B).

The aforementioned findings demonstrated that polymersomes lacking a stimuli-responsive linkage between their hydrophobic and hydrophilic components (MTX-Ps) can successfully deliver drugs to the site of implantation. Adding such a linkage, on the other hand, significantly improves drug release and, as a result, therapeutic effect. Consequently, MTX can be efficiently released and, through its antimetabolite function, impair embryonic development by inhibiting normal cell growth and division.

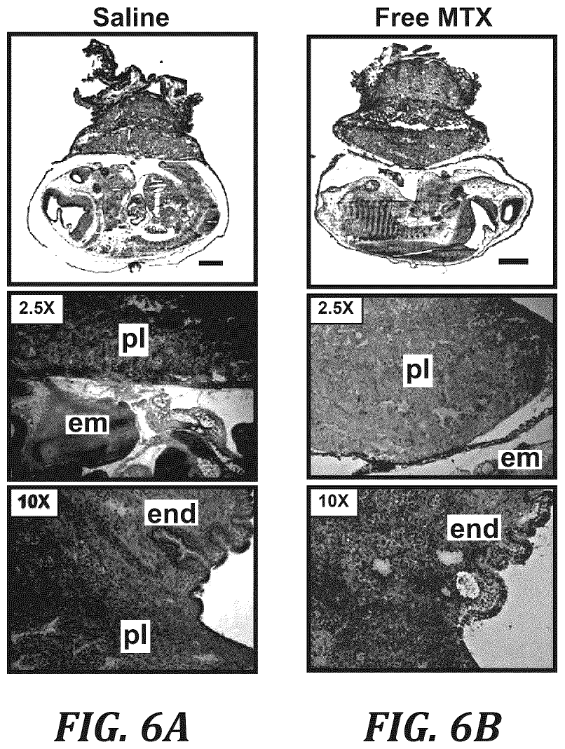

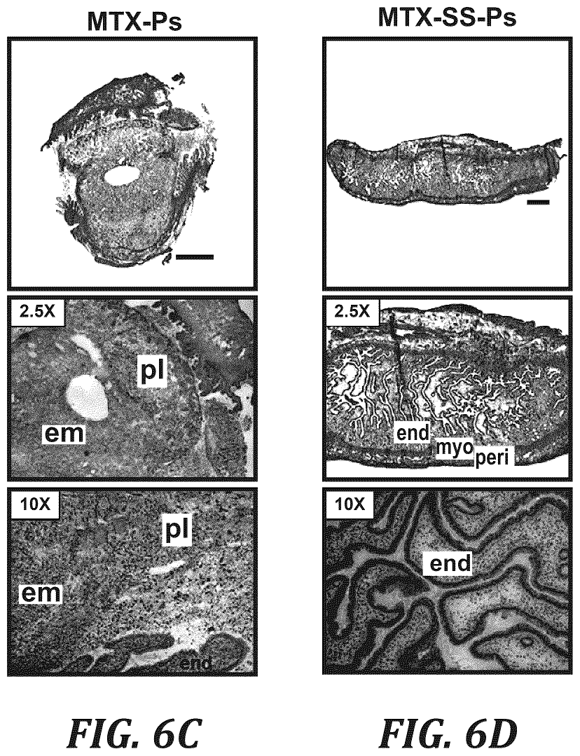

A detailed histopathological assessment of fetoplacental tissues was performed by a board-certified pathologist for the following treatment groups: saline, free MTX, MTX-Ps, or MTX-SS-Ps (FIGS. 6A and 6B). Uteri of mice administered free MTX (1 mg kg-1 MTX, 2×) contained apparently healthy, well-developed fetuses and placentae, with no indications of infarcted tissues or growth retardation, and were histologically similar to those of the saline control group (FIGS. 6A and 6B). Free MTX at this dosing regimen appeared to exert no effect on fetal, placental, or maternal uterine tissues. Conversely, uteri of mice administered MTX-Ps at the same dose (1 mg kg-1 MTX, 2×) contained much smaller, undeveloped, necrotic embryos that lacked nuclear staining, and placentae with severely infarcted regions, primarily in the decidua (FIG. 6C). The maternal uterine tissues of these mice appeared unaffected, with abundant, viable endometrial glands. Notably, administration of MTX-SS-Ps at the same dose (1 mg kg-1 MTX, 2×, at Gd6.5 and Gd8.5) resulted in the demise and complete resorption of all pregnancies by Gd13.5, and uteri of these mice resembled virgin mouse uterus with healthy endometrial glands and stroma (FIG. 6D), as described by a board-certified pathologist.

In a related aspect, the disclosure provides a method of treating an ectopic pregnancy comprising administering to a subject a single dose of a pharmaceutical formulation comprising the polymersome described herein and a pharmaceutically acceptable carrier, wherein the amount of methotrexate, or a pharmaceutically acceptable salt thereof, contained in the administered single dose is sufficient to result in resolution of serum human chorionic gonadotropin (e.g., sustained resolution). As used herein, the phrase “resolution of serum human chorionic gonadotropin” refers to the subject's successful resolution of the growing trophoblast and no longer being pregnant (e.g., sustained resolution).

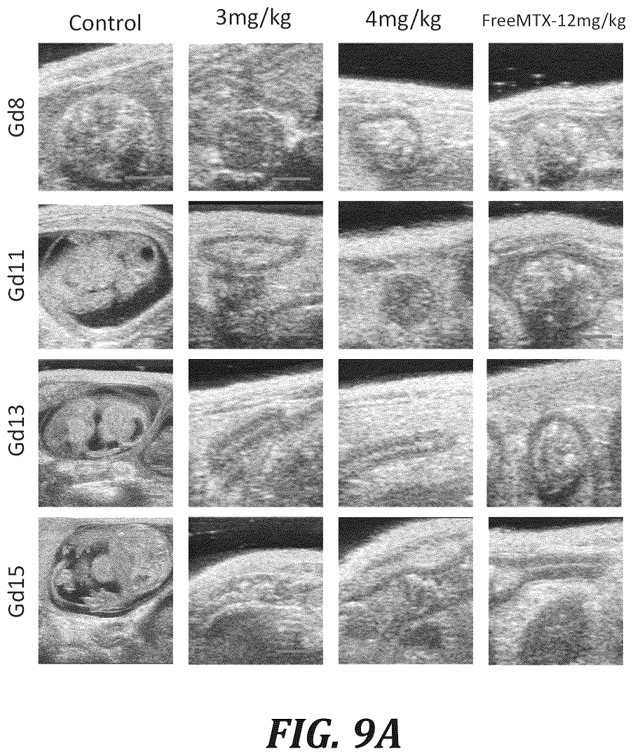

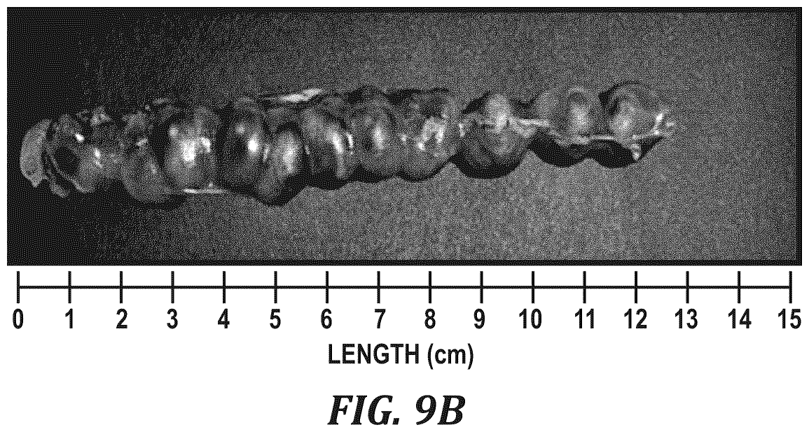

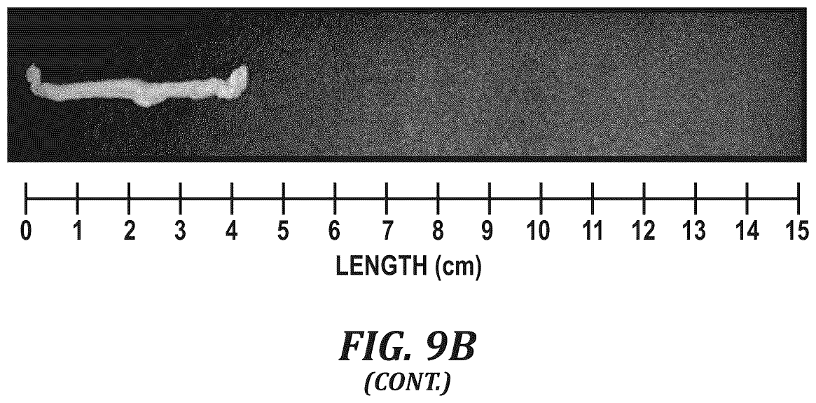

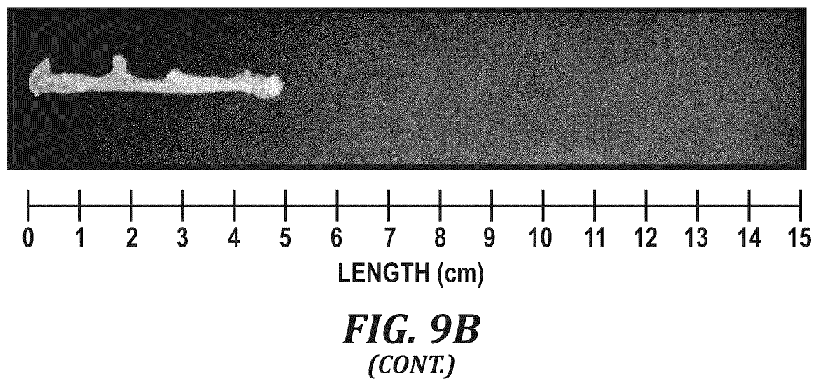

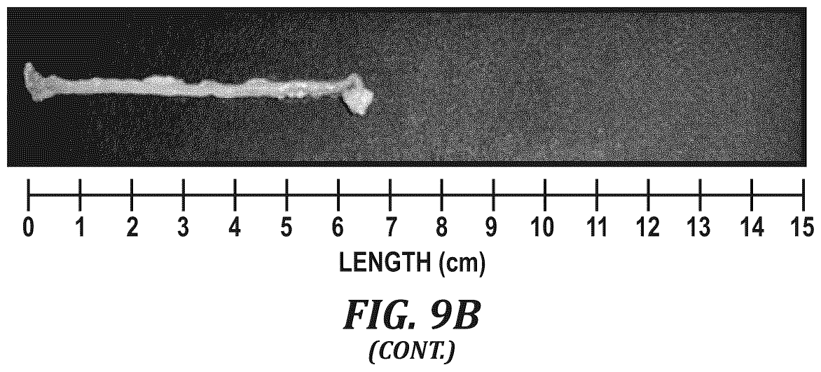

FIGS. 9A and 9B present the ex vivo evaluation of murine pregnancy progress following single dose i.v. administration at Gd6.5. The results show that MTX delivered by the polymersomes described herein induces pregnancy demise in mice, compared to free MTX at the same dose regimen. To achieve the same therapeutic efficacy with free MTX, a 6-fold increase in dosage is required. The MTX-loaded polymersomes described herein act therapeutically at least 2 days faster than free MTX, as demonstrated in mice.

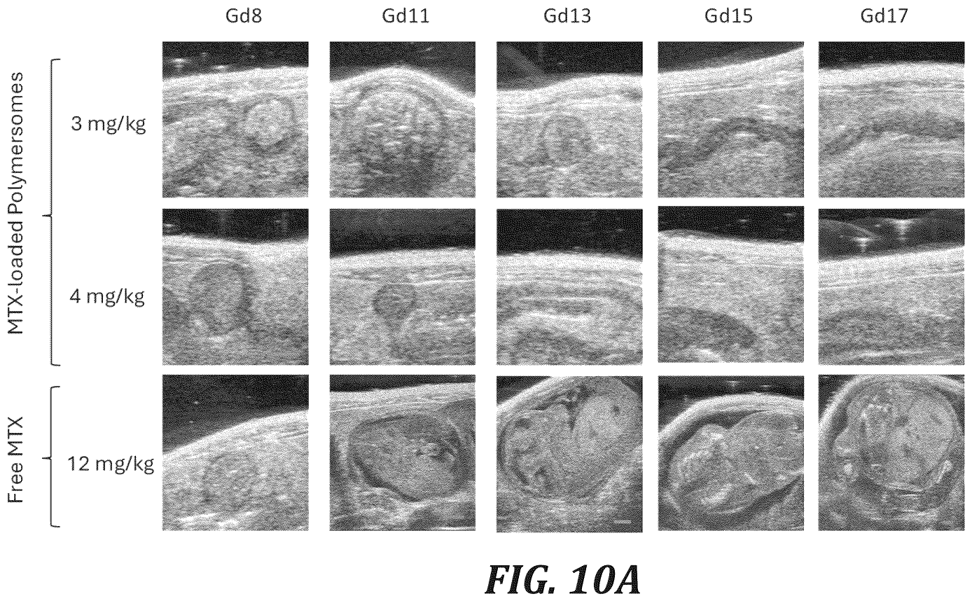

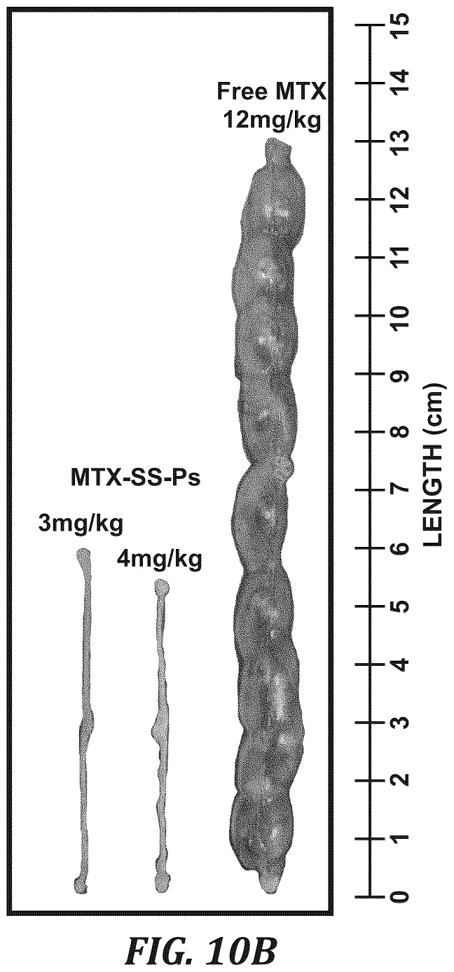

FIGS. 10A and 10B present the ex vivo evaluation of murine pregnancy progress following single dose i.v. administration at Gd8.5. Ultrasound imaging revealed that a single intravenous dose of 3 mg/kg or 4 mg/kg of MTX-SS-Ps with a disulfide bond, administered at Gd6.5, effectively inhibited fetal development after Gd8.5. Pregnancies were completely resorbed by Gd13.5 (FIG. 10A), attributed to improved MTX delivery to the placenta via the nanocarrier. Efficient MTX release was triggered by high GSH concentrations in placental cells. Uteri were resected at Gd15.5, confirming US imaging data (FIG. 10B). When injected at Gd8.5, pregnancies were completely resorbed by Gd15.5 (FIGS. 10A and 10B). In contrast, free MTX at 12 mg/kg showed pregnancy inhibition only at Gd6.5, not at Gd8.5. At the same time, free MTX, administered at 4-fold higher single dose (12 mg/kg), showed pregnancy inhibition at this higher dosage for Gd6.5, and not much for GD8.5.

MTX delivered by polymersomes in a single dose induces pregnancy demise in mice, as opposed to free MTX at the same dose regimen. To achieve the same therapeutic efficacy with free MTX, a 4-fold increase in dosage was administered.

MTX-loaded polymersomes administered in a single dose act therapeutically at least 2 days faster than free MTX, as demonstrated in mice.

Ultrasound imaging revealed that a single intravenous dose of 3 mg/kg or 4 mg/kg of MTX-SS-Ps with a disulfide bond, administered to mice at Gd6.5, effectively inhibited fetal development after Gd8.5. Pregnancies were completely resorbed by Gd13.5 (FIG. 9A) attributed to improved MTX delivery to the placenta via the nanocarrier. Efficient MTX release was triggered by high GSH concentrations in placental cells. Uteri were resected at Gd15.5, confirming US imaging data (FIG. 9B).

When administered to mice at Gd8.5, pregnancies were completely resorbed by Gd15.5 (FIGS. 10A and 10B). In contrast, free MTX at 12 mg/kg showed pregnancy inhibition only at Gd6.5, not at Gd8.5.

At the same time, free MTX, administered at 4-fold higher single dose (12 mg/kg), showed pregnancy inhibition at this higher dosage for Gd6.5, and not much for GD8.5.

MTX-loaded polymersomes administered in a single dose act therapeutically at least 2 days faster than free MTX, as demonstrated in mice.

### Safety Evaluation of MTX Polymersomes and Effects on Subsequent Pregnancy

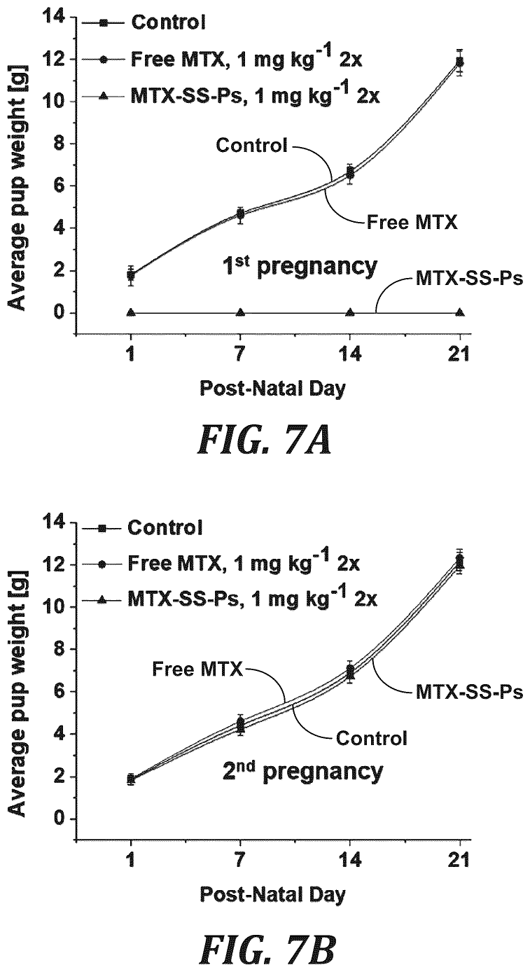

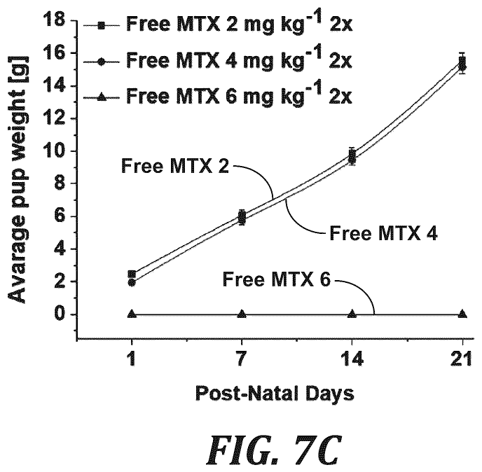

The safety of MTX-SS-Ps for the mother and fetus was assessed. Three groups of pregnant mice at Gd6.5 were i.v. injected with saline, free MTX (1 mg kg-1 MTX at Gd6.5 and 8.5), and MTX-SS-Ps (1 mg kg-1 MTX at Gd6.5 and 8.5). Dams in all groups were allowed to continue gestation following treatment, give birth, nurse pups, and mate again, aiming to assess the success of mating and subsequent pregnancy. Following delivery, individual pup weights from each litter of each group were recorded and compared to evaluate neonatal development. Pups in the saline and free MTX groups were delivered successfully and developed properly, confirming once more that free MTX (at 1 mg kg-1 MTX, 2×) had no effect on the development of the pups. Pup weights in free MTX and saline treatment groups were not statistically different (FIG. 7A). Because administration of MTX-SS-Ps resulted in complete fetal resorption (FIGS. 5A, 5B, 6A, and 6B), no pup weights were reported for this group, and weights for this group are given as 0 g on each post-natal day (pnd) for the duration of the study (FIG. 7A).

These results indicate that a two-dose regimen of free MTX at 1 mg kg-1 had no effect on fetal development, while the same dose administered in the disulfide polymersome formulation was successful at inducing the demise of all pregnancies. Pups from the free MTX group continued to gain weight through pnd 21 (FIG. 7A) and resembled the neonate growth rate in the control group. Pups appeared healthy, ambulatory, actively feeding, well hydrated, and had smooth coats by pnd 21, at which time pups were weaned.

To assess the effects of MTX-SS-Ps on subsequent fertility, dams from the previous experiments (all three groups) were paired with male mice until copulation plugs appeared, and mice were then separated. All dams from the three groups successfully mated and demonstrated similar gestation lengths. All mice subsequently birthed large litters (average 14 pups/litter) of healthy pups, implying that the previous treatment with MTX-SS-Ps had no noticeable effect on the dams' subsequent fertility. Individual pup weights were recorded weekly from pnd 1 to pnd 21 (FIG. 7B), and pups from all three groups appeared healthy and continued to gain weight, with no obvious indications of toxicity resulting from the dams' previous treatment with either free MTX or MTX-SS-Ps, and together with no indicated changes in blood markers in dams (see FIGS. 8A-8C), MTX polymersomes appear to be a safe treatment approach.

Because there was no observable effect of free MTX on fetal development when administered in a two-dose regimen of 1 mg/kg, and in order to compare the efficacy of MTX-SS-Ps to that of free drug, a dose of free MTX was identified that would exert an inhibitory effect on fetal development. Pregnant mice were administered a two-dose regimen of free MTX at 2, 4, or 6 mg/kg, with a single i.v. injection on Gd6.5 and Gd8.5. Pregnant mice from the 2 and 4 mg/kg groups continued to increase in size and ultimately birthed litters (average 11 pups), while dams administered 6 mg/kg free MTX appeared non-pregnant throughout the study and did not birth any pups (FIG. 7C). These results indicate that the MTX-SS-Ps formulation was capable of terminating pregnancy at a dose 6-fold less than that of free MTX.

As noted above, EP, the abnormal implantation of an embryo in the uterus or sites outside of the uterine cavity, can lead to rupture and hemorrhage if not identified and treated in a timely manner, and is the primary contributor to maternal morbidity and mortality in the first trimester. The disclosure provides GSH-responsive polymersomes that efficiently encapsulate and preferentially deliver MTX to implantation sites while ensuring nearly complete triggered drug release. The MTX-loaded polymersomes were produced with a size of less than 40 nm and a nearly neutral surface charge and released 95% of MTX during 20 hours. These polymersomes successfully accumulate in mouse implantation sites within 24 hours of administration at both Gd6.5 and Gd12.5, as shown by fluorescence and PA imaging in addition to histological analysis. The developed MTX nanomedicine described herein effectively suppressed fetal development and completely eliminated conceptuses in a pregnant mouse model at a six-fold lower dose than free MTX. Because the developed formulation is biocompatible, and dams successfully conceived and birthed healthy pups following a prior complete pregnancy demise induced by MTX-SS-Ps, MTX nanomedicine appears to be a safe therapeutic method. These results indicate that the developed MTX nanomedicine can address several challenges associated with the currently used free MTX regimen for ectopic pregnancy management, including insufficient accumulation of the drug at the ectopic implantation site, a high failure rate of MTX treatment and various side effects. The MTX nanomedicine described herein can improve the clinical management of ectopic pregnancy, minimize side effects associated with free MTX, and consequently reduce associated mortality rates and costs.

In a second aspect, the present disclosure provides methotrexate-loaded polymersomes and their use for treating gestational trophoblastic neoplasia (GTN) (e.g., choriocarcinoma (CC).

Gestational trophoblastic disease (GTD) is benign abnormally proliferated trophoblastic tissue, such as hydatidiform molar pregnancy. GTD can become invasive and transform into malignant gestational trophoblastic neoplasia (GTN). GTN encompasses a spectrum of conditions, including choriocarcinoma (CC), placental-site trophoblastic tumors, invasive moles, and epithelioid trophoblastic tumors, and it can arise from molar, ectopic, and term pregnancies, miscarriage, or abortion. CC, the most common malignant GTN, is distinguished by trophoblastic hyperplasia and anaplasia, significant hemorrhage and necrosis, and the absence of chorionic villi. CC is a highly aggressive form of trophoblastic cancer and has a significant incidence in the US, with 2 to 7 cases reported per 100,000 pregnancies. Although CC responds to chemotherapy, the five-year overall survival rate ranges from 82% to 92%. Currently, diagnosis of CC relies on tumor biopsy, conducting histological evaluations, and blood tests to monitor serum levels of human chorionic gonadotropin (hCG). Gestational CC, which is the most aggressive form of trophoblast cancer, encompasses 5% of all GTN cases. As a form of malignant neoplasia, gestational CC readily metastasizes via hematogenous spread to various organs, such as the lungs, kidneys, brain, liver, breasts, bones, gastrointestinal tract, and lymph nodes.

CC treatment varies based on disease severity, ranging from a single dose of methotrexate (MTX) or actinomycin-D (Act-D) for low-risk cases to a comprehensive regimen of multi-dose chemotherapy, radiation, and surgery for high-risk metastatic instances. Due to the aggressive and metastatic nature of CC, treatment frequently requires multiple doses of MTX. MTX is administered at a dosage range of 30-50 mg/m², typically on a weekly basis, with a monotherapy success rate ranging between 57-90%. While MTX is favored for CC treatment, its poor tumor specificity in standard applications can cause severe side effects such as liver and kidney toxicity, particularly in multi-dose chemotherapy for metastatic CC. Given MTX's role as the mainstay treatment for CC, the critical goal now is to enhance its effectiveness, including faster response times, while simultaneously minimizing side effects.

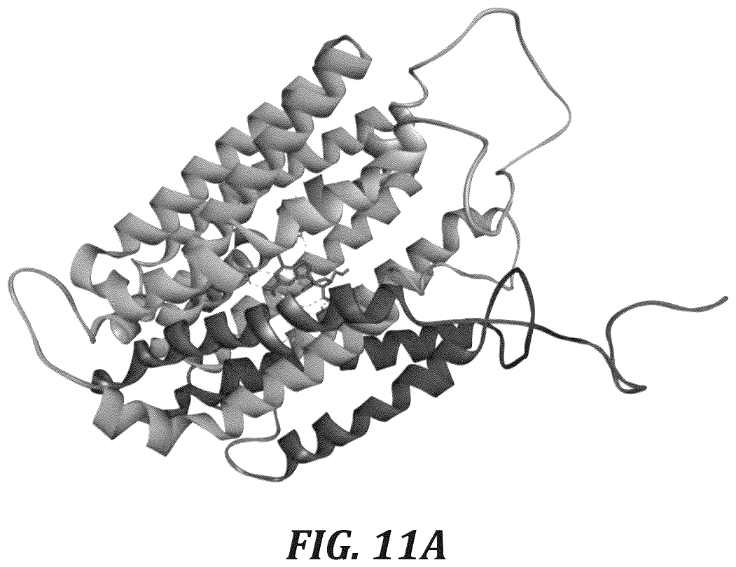

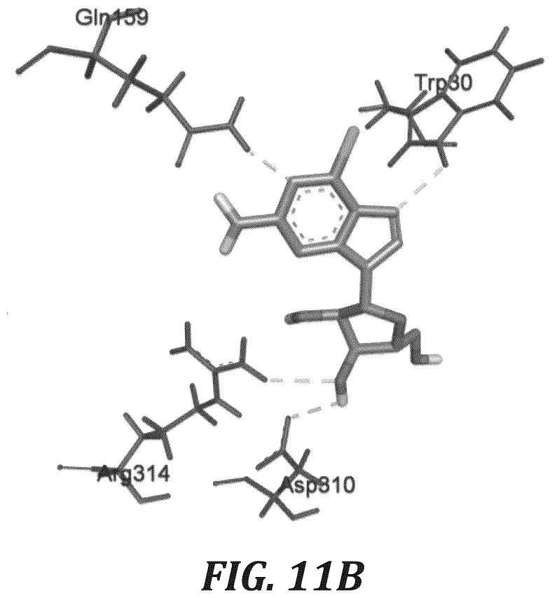

The present disclosure employs a specially designed nanoplatform that ensures precise drug delivery and release directly into CC tumors. The present disclosure provides an MTX-loaded polymersome (Ps) nanoplatform functionalized with guanosine (Gn) to target the equilibrative nucleoside transporter 1 (ENT-1) (FIGS. 11A and 11B), which is overexpressed in JEG-3 choriocarcinoma cells, to improve CC tumor accumulation. In addition, the incorporation of a stimuli-responsive linker ensures selective release of MTX within the intratumoral environment of the CC. The delivery of MTX within biocompatible Ps nanocarriers, equipped with triggered intracellular release linkages and targeting ligands for CC cells, enables rapid and efficient MTX delivery to CC tumors.

Gestational trophoblastic neoplasia (GTN) represents a spectrum of malignant conditions arising from abnormally proliferated trophoblastic tissue, which can develop from molar pregnancies, ectopic pregnancies, term pregnancies, miscarriages, or abortions. encompasses several distinct pathological entities, including choriocarcinoma (CC), placental-site trophoblastic tumors, invasive moles, and epithelioid trophoblastic tumors. The current standard of care for GTN relies primarily on methotrexate (MTX) or actinomycin-D for low-risk cases, with multi-agent chemotherapy regimens reserved for high-risk metastatic disease. However, the poor tumor specificity of conventional MTX administration often necessitates multiple doses and can result in significant systemic toxicity, particularly hepatic and renal adverse effects.

Among the GTN subtypes, choriocarcinoma (CC) presents as the most common malignant form and is distinguished by its highly aggressive behavior, characterized by trophoblastic hyperplasia and anaplasia, extensive hemorrhage and necrosis, and the absence of chorionic villi. Gestational CC, representing approximately 5% of all GTN cases, is particularly notable for its propensity for rapid hematogenous metastasis to distant organs including the lungs, kidneys, brain, liver, and bones. Given that CC cells overexpress the equilibrative nucleoside transporter 1 (ENT-1), this molecular target offers a promising avenue for developing targeted therapeutic delivery systems that could enhance MTX efficacy while minimizing off-target effects.

In another aspect, the disclosure provides a method for treating gestational trophoblastic neoplasia (GTN) in a subject (e.g., human). In certain embodiments, the method comprises administering to a subject in need thereof, a therapeutically effective amount of a methotrexate-containing polymersome, wherein the methotrexate-containing polymersome comprises methotrexate and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block, wherein the polymersome has a bilayer structure, with a hydrophilic interior core, hydrophobic layer and a hydrophilic outer shell formed by the hydrophilic PEG block, wherein methotrexate, or a pharmaceutically acceptable salt thereof, is encapsulated in the hydrophilic interior core, and wherein the polymersome comprises one or more targeting moieties that bind to receptors expressed on the surface of GTN cells.

In certain embodiments, the targeting moieties are guanosine moieties that target equilibrative nucleoside transporter 1 (ENT-1) expressed on the surface of the neoplasia cells.

In a related aspect, the disclosure provides a method for treating gestational choriocarcinoma (CC) in a subject (e.g., human). In certain embodiments, the method comprises administering to a subject in need thereof a therapeutically effective amount of a methotrexate-containing polymersome, wherein the methotrexate-containing polymersome comprises methotrexate and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block, wherein the polymersome has a bilayer structure, with a hydrophilic interior core, hydrophobic layer and a hydrophilic outer shell formed by the hydrophilic PEG block, wherein methotrexate, or a pharmaceutically acceptable salt thereof, is encapsulated in the hydrophilic interior core, and wherein the polymersome comprises one or more guanosine moieties to target equilibrative nucleoside transporter 1 (ENT-1) expressed on the surface of choriocarcinoma cells (e.g., JEG-3).

In certain embodiments, therapeutically effective amounts of methotrexate (or equivalent pharmaceutically acceptable salt thereof) administrated by the methods range from about 1 mg/m² to about 50 mg/m² (methotrexate/subject surface area). In other embodiments, the amount of methotrexate (or equivalent pharmaceutically acceptable salt thereof) administrated by the methods range from about 10 mg/m² to about 50 mg/m², from about 10 mg/m² to about 25 mg/m², from about 10 mg/m² to about 20 mg/m², from about 10 mg/m² to about 15 mg/m² (methotrexate/subject surface area). In one embodiment, the amount of methotrexate (or equivalent pharmaceutically acceptable salt thereof) administrated by the method is about 12.5 mg/m² (methotrexate/subject surface area).

In certain embodiments, therapeutically effective amounts of methotrexate (or equivalent pharmaceutically acceptable salt thereof) administrated by the methods range from about 1 mg/m² to about 200 mg/m² (methotrexate/subject surface area).

In certain embodiments of the method, therapeutically effective amounts of methotrexate (or equivalent pharmaceutically acceptable salt thereof) administrated by the methods range from about 0.1 mg/kg to about 2.0 mg/kg (methotrexate/subject body weight). In certain of these embodiments, therapeutically effective amounts of methotrexate administrated by the methods range from about 0.2 to about 1.9, about 0.3 to about 1.8, about 0.4 to about 1.7, about 0.5 to about 1.6, about 0.6 to about 1.5, about 0.7 to about 1.4, about 0.8 to about 1.3, about 0.9 to about 1.2, or about 0.9 to about 1.1 mg/kg (methotrexate/subject body weight). In one embodiment the therapeutically effective amount of methotrexate (or equivalent pharmaceutically acceptable salt thereof) administrated by the methods is about 1.1 mg/kg (methotrexate/subject body weight).

In certain embodiments, therapeutically effective amounts of methotrexate (or equivalent pharmaceutically acceptable salt thereof) administrated by the methods range from 0.1 mg/kg to about 4.0 mg/kg (methotrexate/subject body weight).

It will be appreciated that the dose and number of doses administered will be determined based on the subject. In certain embodiments, the number of doses of the methotrexate-containing polymersome is from 1 to 10. In other embodiments, the number of doses of the methotrexate-containing polymersome is from 3 to 8. In a further embodiment, the number of doses of the methotrexate-containing polymersome is about 6.

In the methods described herein, the methotrexate-containing polymersome is systemically administered by, for example, intravenous injection. For such administration, the polymersomes described herein are formulated as pharmaceutical compositions with a pharmaceutically acceptable carrier or diluent. Suitable carriers include solutions for injection, such as saline and dextrose solutions.

In certain embodiments, the methotrexate-containing polymersome described herein is in lyophilized form that may be reconstituted at the site for administration (e.g., patient's bedside) with an injectable carrier to provide a solution for administration.

The following description is directed to representative methotrexate-containing polymersomes and their use in treating choriocarcinoma (CC).

### Development of Guanosine (Gn)-Modified Polymersomes

As described above, a nano-Ps formulation loaded with MTX successfully delivered and released MTX directly to placental cells at the implantation site in mice. This delivery approach required a dose six times lower than free MTX to achieve the same level of therapeutic efficacy. Ps, which are self-assembled bilayer polymeric vesicles made of amphiphilic diblock copolymers, are capable of encapsulating water-soluble molecules, including MTX sodium salt, in their hydrophilic core and lipid-soluble molecules, including contrast agents or majority of small molecule drugs, in their hydrophobic bilayer. As described herein, an MTX-loaded Ps formulation was prepared using microfluidic technology (NanoAssemblr™ Ignite™) to generate stable and uniform polymersomes. Ps were crafted using an amphiphilic diblock copolymer, featuring a hydrophobic PCL (5k) block to ensure bilayer stability and a PEG (2k) block to establish a hydrophilic core and a water-soluble shell. Aiming to achieve rapid release of MTX, Ps made from an amphiphilic copolymer containing a disulfide bond between PEG and PCL blocks (PEG-SS-PCL) were used to enable selective release of drug cargo inside cells. Once inside CC cells, the polymersome's internal disulfide bond undergo rapid reduction due to the elevated concentration of glutathione (GSH) within the intracellular environment. This process triggers a disulfide exchange reaction with GSH, leading to the breakdown of the nanocarriers and the efficient release of the drug.

To improve the delivery of nanocarrier-loaded MTX to CC tissue, ENT-1 was identified as an effective target for guiding the developed drug delivery system to CC cells. ENT-1, one of the membrane transporters in human placenta, plays a crucial role in supplying nutrients and transporting small drug molecules and is overexpressed in CC cells (JEG-3). ENT-1 transports nucleosides, such as adenosine, suggesting that functionalizing the developed MTX-loaded Ps with adenosine derivatives can enhance its delivery to CC cells with the help of the ENT-1 transporter. To identify the optimal targeting ligand for the ENT1 transporter, twenty (20) adenosine derivatives were evaluated to identify a derivative with the highest binding affinity. See Table 2. The ENT-1 crystal structure was obtained from Protein Data Bank (PDB, ID: 6OB7). Next, to identify the derivative with the highest binding affinity towards ENT-1, a molecular docking analysis was performed using AutoDock Vina to evaluate binding free energy between the ligand and target protein. The most negative binding free energy (ΔG<0) of a system is the best indicator of a stable ligand-protein complex. As a result, 6-chloro-guanosine (Gn), with a binding affinity of −8.4 kcal/mol, was selected as an optimal ligand to bind to ENT-1. The protein-ligand complex was visualized by the Biovia Discovery Studio Visualizer (FIG. 11B), demonstrating that Gn makes 4 hydrogen bonds with glutamine 159, tryptophan 30, arginine 314, and aspartate 310 residues within the binding cavity of ENT-1.

TABLE 2
Binding affinity (kcal/mol) of selected
adenosine derivatives for ENT-1 protein.

| | | Binding affinity | Compound |
|---|---|---|---|
| Number | Ligand | (kcal/mol) | CID |
| 1 | Adenosine | −7.3 | 60961 |
| 2 | Amino-adenosine | −8.2 | 72200 |
| 3 | Amino-purine riboside | −8.1 | 11065406 |
| 4 | Chloro-5-Deoxyadenosine | −7.4 | 5327118 |
| 5 | Chloro-adenosine | −7.8 | 8974 |
| 6 | Chloro-guanosine | −8.4 | 102197 |
| 7 | Chloro-tubercidin | −7.8 | 97453 |
| 8 | Cordyceptin | −7.1 | 6303 |
| 9 | Deaza-adenosine | −7.2 | 23190 |
| 10 | Deoxy-2-fluoroadenosine | −7.8 | 447864 |
| 11 | Deoxy adenosine | −7.3 | 439182 |
| 12 | Deoxy tubercidin | −7.5 | 3006222 |
| 13 | Fluoro-5-deoxyadenosine | −7.4 | 100253 |
| 14 | Fluoro-adenosine | −7.6 | 8975 |
| 15 | Formycin A | −7.9 | 135449281 |
| 16 | Guanosine | −8.0 | 135398635 |
| 17 | Thioadenosine | −6.9 | 128924 |
| 18 | Thioguanosine | −6.8 | 2724387 |
| 19 | Tubercidin | −7.6 | 6245 |
| 20 | Xylosyl-adenine | −7.3 | 160739 |

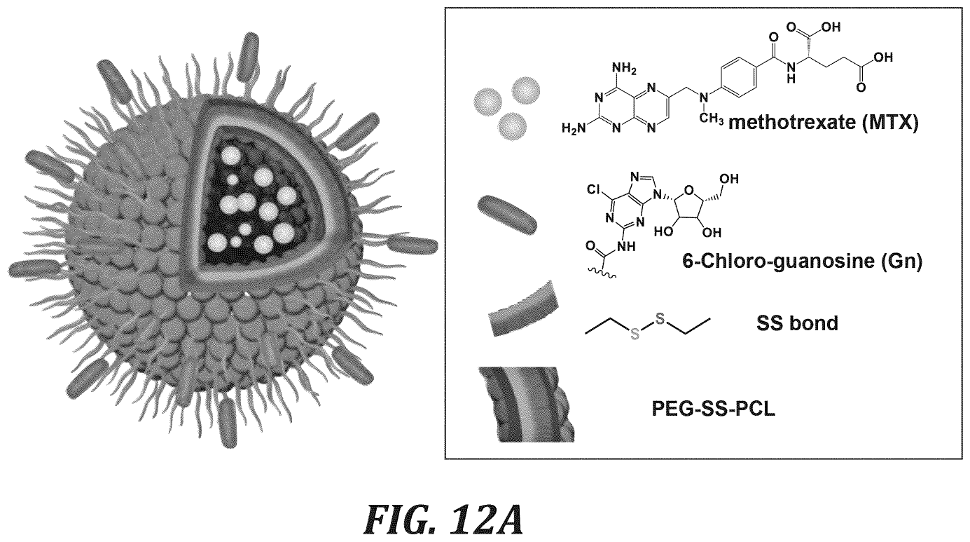

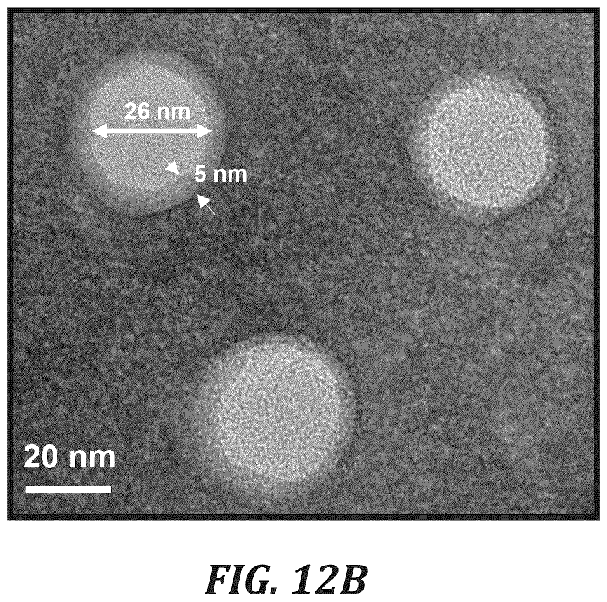

To functionalize the Ps with Gn, the carboxylic group of PCL-SS-PEG (PCL-SS-PEG-COOH) was conjugated to the amine group of Gn, generating a peptide bond. The conjugation yield of this reaction was about 99%, as determined by the Pierce™ Quantitative Fluorometric Peptide Assay kit. To formulate Gn-modified Ps loaded with the MTX (Gn-MTX@SS-Ps, FIG. 12A), a mixture of the prepared Gn-PEG-SS-PCL and methoxy PEG-SS-PCL (CH₃O-PEG-SS-PCL) was used at a 1:9 ratio. Non-targeted Ps (MTX@SS-Ps), formulated using only CH₃O-PEG-SS-PCL without the targeting moiety, was prepared as a control. The constructed Gn-MTX@SS-Ps (FIG. 12A) has a spherical shape (FIG. 12B), nearly neutral surface charge (−2.48±0.21, n=3), uniform distribution (PDI of 0.11), and a hydrodynamic size of about 42 nm. Transmission electron microscopy (TEM) confirms the Gn-MTX@SS-Ps's double-layer structure with about 26 nm in diameter of the inner hydrophilic sphere and about 5 nm outer hydrophobic layer (FIG. 12B). These Ps allowed loading 30 mg of MTX per 10 mg of polymer in 1 mL of solution, for both non-targeted and targeted polymersomes. Previously, it was confirmed that MTX@SS-Ps effectively releases MTX in solution, exceeding 80% drug (MTX) release at 1 mM GSH. Since cytosolic concentrations of GSH are between 1 to 10 mM, the high MTX release observed from MTX@SS-Ps at 1 mM GSH was promising. Storing Gn-modified MTX-loaded polymersomes at 4° C. for 4 weeks resulted in only minimal size changes, indicating good stability and extended shelf life for this formulation.

### Evaluation of ENT-1 Targeted Gn-Modified Polymersomes In Vivo

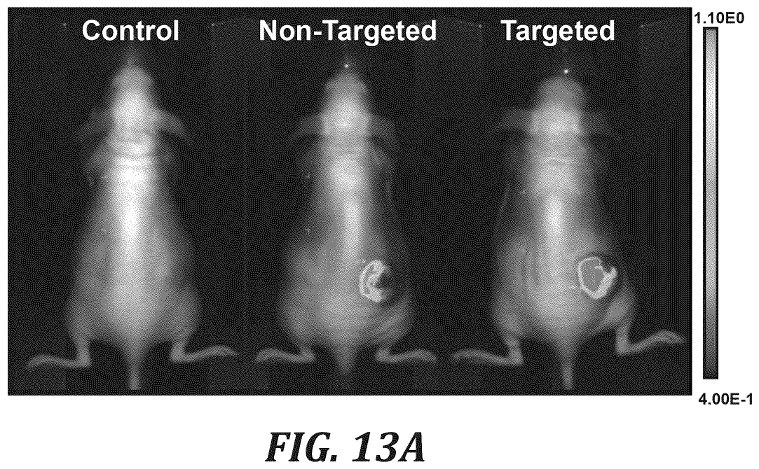

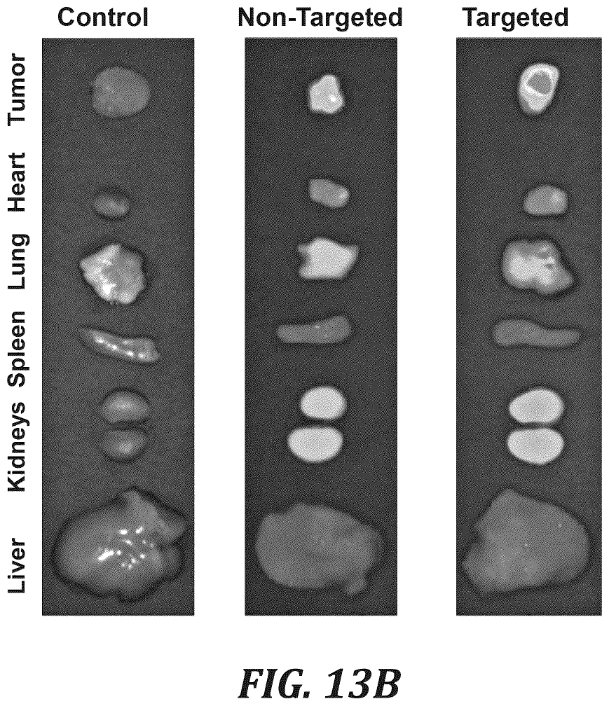

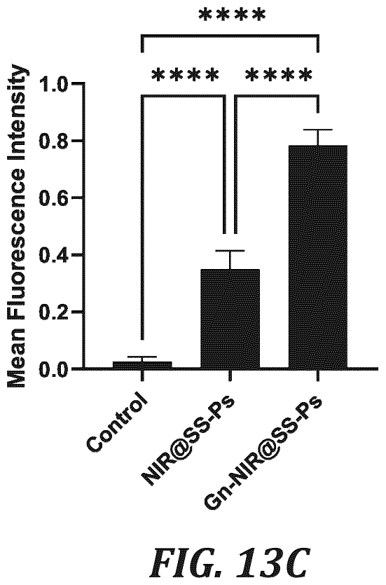

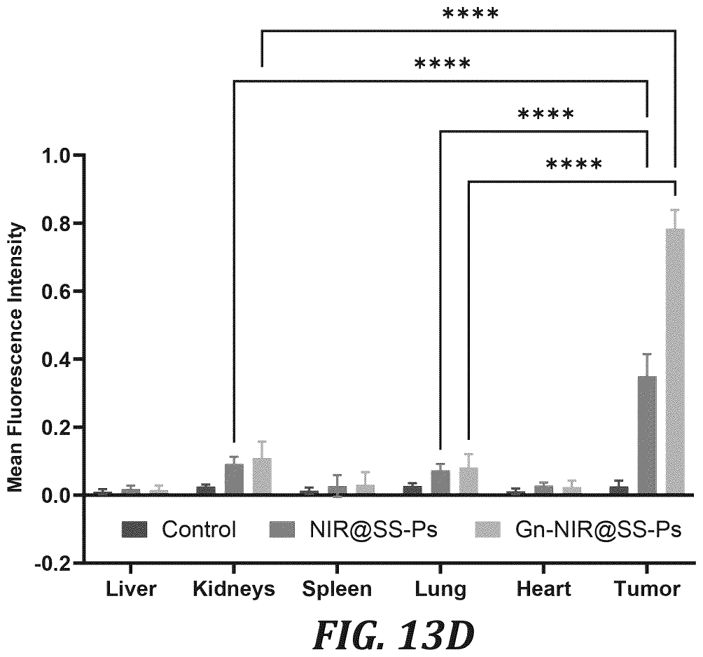

To determine if Gn-modification enhances the uptake of Ps by CC tumors in vivo, the biodistribution of non-targeted and ENT-1-targeted Ps was evaluated using fluorescence imaging. This was achieved by monitoring the fluorescence of the loaded NIR dye. At 12 hours post-injection (i.v.), both NIR@SS-Ps and Gn-NIR@SS-Ps produce a prominent fluorescence signal in subcutaneous JEG-3 tumors (FIGS. 13A and 13B). The ENT-1-targeted polymersome outperformed the non-targeted ones in terms of tumor accumulation (FIG. 13C), as evidenced by the fluorescence intensity that was 2-fold greater in the tumors treated with the targeted Ps (Gn-NIR@SS-Ps) in comparison with the non-targeted group (NIR@SS-Ps). Fluorescence imaging of resected organs revealed near complete clearance of NIR@SS-Ps and Gn-NIR@SS-Ps from major organs (FIGS. 13A and 13B). While some accumulation of Ps was observed in kidneys and lungs, the signal was comparable to background levels, and there was no significant difference in fluorescence intensity between these organs and those of control mice administered saline (FIG. 13D). The fluorescence signal of non-targeted Ps in kidneys was 25% of that observed in tumors. Furthermore, the difference was even more prominent with the Gn-NIR@SS-Ps, with an almost 7-fold (86%) increase in fluorescence intensity in tumors compared to kidneys (FIG. 13D). These biodistribution data indicate a preferential uptake and retention of the Ps in the CC tumors enhanced by ENT-1-targeting, suggesting therapeutic promise for improved antineoplastic efficacy and substantially minimized off-target side effects.

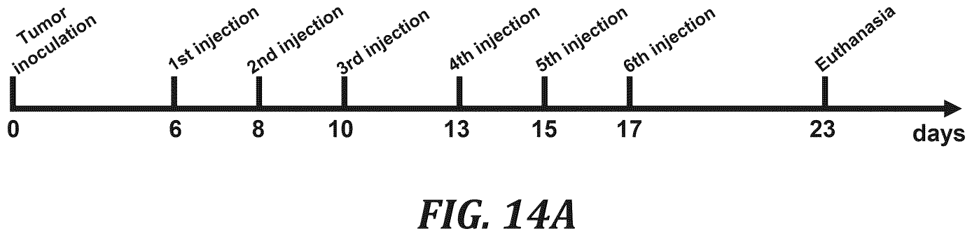

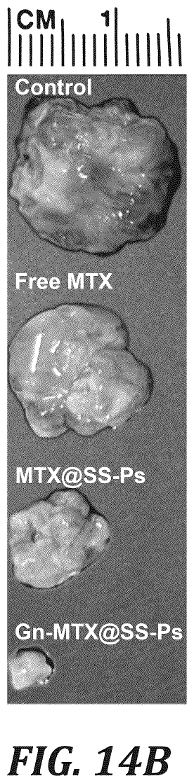

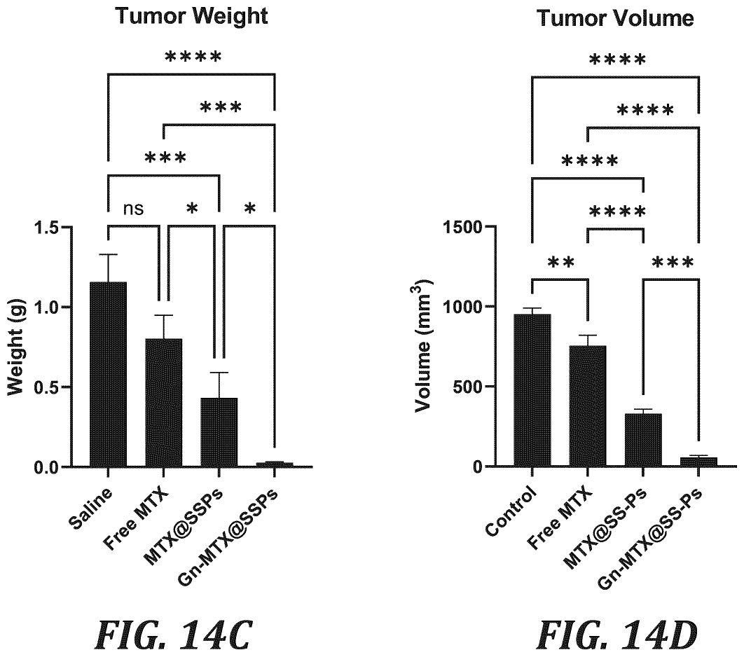

Given the promising in vivo biodistribution data, the antitumor activity of ENT1-targeted Gn-MTX@SS-Ps was evaluated in a JEG-3 tumor mouse model. When subcutaneous tumors reached approximately 100 mm³ in volume, the mice were treated with Gn-MTX@SS-Ps and controls (free MTX, MTX@SS-Ps, and saline) every other day (10 mg kg⁻¹, for a total of 6 doses, FIG. 14A). Tumors in both treated and control mice were monitored for an additional six days following the last injection. Throughout the 23-day study period, using a consistent dose regimen (10 mg kg⁻¹ MTX per dose), tumors in the saline and free MTX groups continued to grow, whereas the non-targeted MTX@SS-Ps led to a deceleration of tumor progression. Importantly, the targeted Gn-MTX@SS-Ps halted tumor growth. After euthanasia, the tumor volumes, along with tumor weight, in the Gn-MTX@SS-Ps treatment group were significantly reduced, by 95% compared to the non-treated (saline) group (FIGS. 14B-14D). Tumor growth halted after the 2^{nd} dose and continued to decrease with subsequent doses in the Gn-MTX@SS-Ps treatment group, whereas tumors in other groups continued to grow at varying rates (FIG. 14D). In comparison, the non-targeted MTX@SS-Ps reduced tumor growth by 65%, and free MTX by only 20%, relative to the saline control group. The tumors treated with Gn-MTX@SS-Ps exhibited the most potent treatment effect, demonstrating approximately 6-fold higher antitumor efficacy compared to the non-targeted MTX@SS-Ps.

The promising antitumor efficacy was primarily due to the enhanced cellular uptake of ENT-1 targeted polymersomes and the effective release of MTX within the tumor, facilitated by efficient intracellular disulfide bond cleavage in the presence of GSH. These findings underscore the clear advantage of ENT1 targeting and tumor-sensitive drug release in chemotherapy for the treatment of CC.

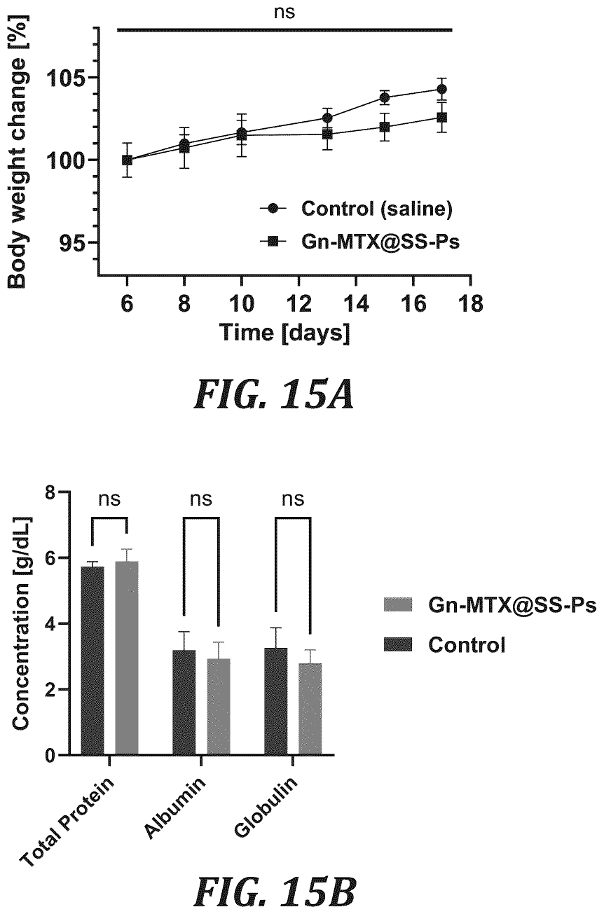

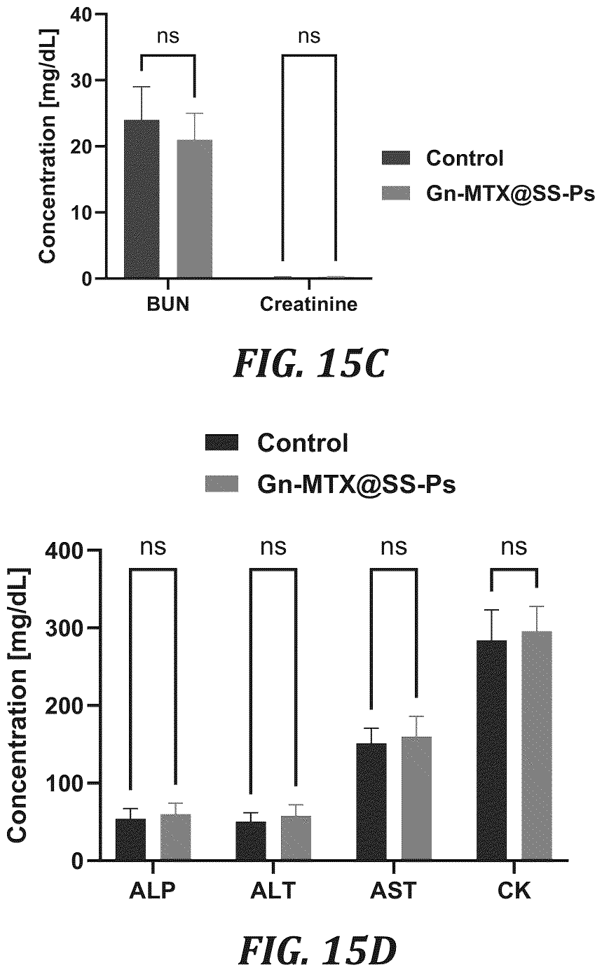

The mice treated with Gn-MTX@SS-Ps showed no signs of toxicity, weight loss, or mortality throughout the study (FIG. 15A). Blood chemistry analysis further revealed no significant differences in serum levels of blood urea nitrogen (BUN), alkaline phosphatase (ALP), alanine aminotransferase (ALT), aspartate transaminase (AST), creatine kinase (CK), proteins, and electrolytes among animals after 6 doses (10 mg kg⁻¹) of targeted Gn-MTX@SS-Ps (FIGS. 15B-15D). Therefore, the ENT-1 targeting moiety does not compromise the safety profile of the polymersome.

As described herein, methotrexate (MTX)-loaded polymersomes (Ps) targeted to the ENT-1 transporter overexpressed in choriocarcinoma cells enhance treatment efficacy of MTX therapy and minimizes systemic side effects. Functionalizing Ps with guanosine (Gn) significantly improved cellular uptake and efficient drug release within the tumor, driven by intracellular disulfide bond cleavage in the presence of glutathione. In vivo fluorescence imaging showed substantial accumulation of targeted Ps in CC tumors with minimal distribution in major organs. In a mouse model, ENT-1 targeted Ps demonstrated a 95% reduction in tumor volume, exhibiting approximately six-fold higher antitumor efficacy compared to non-targeted Ps. As described herein, ENT-1 targeted polymersomes for improved chemotherapy in choriocarcinoma treatment, with enhanced drug delivery and reduced systemic toxicity. As demonstrated herein, ENT-1 targeted MTX-loaded polymersomes significantly improve drug delivery and anti-tumor efficacy in JEG-3 mouse model, for application in choriocarcinoma treatment.

### Experimental: Representative Polymersomes and Related Methods for Treating Ectopic Pregnancy

Materials. PEG(2k)-PCL(5k) (methoxy poly(ethylene glycol)-b-poly(ε-caprolactone) copolymer was purchased from Akina Inc (West Lafayette, IN). PEG(2k)-SS-PCL(5k) (methoxy poly(ethylene glycol)-b-disulfide-poly(ε-caprolactone) copolymer was synthesized by Ruixibiotech (Xian, China). NIR dye (silicon 2,3-naphthalocyanine bis (trihexylsilyloxide)) was purchased from Alfa Chemistry (Ronkonkoma, NY, USA). USP-grade methotrexate sodium salt (MTX) was obtained from OHSU Pharmacy (Hikma Pharmaceuticals USA Inc., Berkeley Heights, NJ). MilliporeSigma (Milwaukee, WI), Fisher Scientific Inc. (Hampton, NH) and VWR International, LLC (Radnor, PA) provided other common chemicals and supplies.

Preparation of polymersomes. MTX-loaded polymersomes were prepared using PEG-PCL and PEG-SS-PCL copolymers via a microfluidic mixing method (see, e.g., M. I. Confeld, B. Mamnoon, L. Feng, H. Jensen-Smith, P. Ray, J. Froberg, J. Kim, M. A. Hollingsworth, M. Quadir, Y. Choi, S. Mallik, Mol. Pharm. 2020, 17, 2849; and F. Karandish, B. Mamnoon, L. Feng, M. K. Haldar, L. Xia, K. N. Gange, S. You, Y. Choi, K. Sarkar, S. Mallik, Biomacromolecules 2018, 19, 4122). Copolymer (10.0 mg) and MTX (15.0 mg) were dissolved in 1 mL of acetone and 1 mL of saline, respectively. Then, obtained solutions were loaded into two Hamilton glass syringes, and a Harvard DDS dual independent channel syringe pump (Holliston, MA) was used for mixing aqueous and organic phases at a flow rate of 2.2 mL min-1 through a microfluidic mixer chip (Precigenome, San Jose, CA). Next, the organic solvent was evaporated, and an Amicon Ultra-4 centrifugal filter unit (Merck Millipore, MWCO: 30 KDa, 10 min, 5500 rpm) was used to remove the unencapsulated drug. Finally, the MTX-loaded nanoparticle solution was passed through a 0.22 μm filter for the final purification to produce MTX-Ps or MTX-SS-Ps. NIR dye (naphthalocyanine derivative) was loaded into polymersomes using a similar method; copolymer (10.0 mg, PEG-PCL or PEG-SS-PCL) and NIR dye (0.3 mg) were dissolved in tetrahydrofuran (THF, 1 mL total), and NIR-Ps or NIR-SS-Ps were generated using a glass microfluidic mixer chip (Precigenome, San Jose, CA) compatible with THF.

Characterization of polymersomes. The size, surface charge, and polydispersity of the polymersomes were determined using a Malvern ZetaSizer (Worcestershire, UK). Cryogenic Transmission Electron Microscopy (Cryo-TEM) (Thermo Fisher Scientific, Waltham MA) was used for assessing the morphology of nanoparticles (see, e.g., C. Schumann, D. X. Nguyen, M. Norgard, Y. Bortnyak, T. Korzun, S. Chan, A. S. Lorenz, A. S. Moses, H. A. Albarqi, L. Wong, K. Michaelis, X. Zhu, A. W. G. Alani, O. R. Taratula, S. Krasnow, D. L. Marks, O. Taratula, Theranostics 2018, 8, 5276; B. Mamnoon, L. Feng, J. Froberg, Y. Choi, V. Sathish, S. Mallik, Mol. Pharm. 2020, 17, 4312; and B. Mamnoon, L. Feng, J. Froberg, Y. Choi, V. Sathish, O. Taratula, O. Taratula, S. Mallik, ACS Omega 2021, 6, 27654; and B. Mamnoon, J. Loganathan, M. I. Confeld, N. De Fonseka, L. Feng, J. Froberg, Y. Choi, D. M. Tuvin, V. Sathish, S. Mallik, ACS Appl. Bio. Mater. 2021, 4, 1450). Loading of MTX in the polymersome nanoparticles was evaluated at 302 nm by preparing a calibration curve of serial MTX solutions (see, e.g., O. L. Lawani, O. B. Anozie, P. O. Ezeonu, Int. J. Womens Health 2013, 5, 515; F. A. Taran, K. O. Kagan, M. Hübner, M. Hoopmann, D. Wallwiener, S. Brucker, Dtsch. Arztebl. Int. 2015, 112, 693; and K. T. Barnhart, W. Guo, M. S. Cary, C. B. Morse, K. Chung, P. Takacs, S. Senapati, M. D. Sammel, Obstet. Gynecol. 2016, 128, 504) using high-performance liquid chromatography (HPLC, Shimadzu, Japan) with an Agilent ZORBAX C-18 column (3.5 μm, 4.6×75 mm) at a flow rate of 1 mL min-1 and a mobile phase comprised of acetonitrile/water (35:65 v/v %) stabilized with 0.1% trifluoroacetic acid. The stability of MTX-encapsulated polymersomes in saline, stored at 4° C., was evaluated for 8 weeks by examining the size and polydispersity index of the nanoparticles (see. e.g., F. A. Taran, K. O. Kagan, M. Hübner, M. Hoopmann, D. Wallwiener, S. Brucker, Dtsch. Arztebl. Int. 2015, 112, 693). Drug (MTX) release from the polymersomes was monitored in the presence of different concentrations of glutathione (GSH) in phosphate-buffered saline (PBS) as a function of time, as described in F. Karandish, B. Mamnoon, L. Feng, M. K. Haldar, L. Xia, K. N. Gange, S. You, Y. Choi, K. Sarkar, S. Mallik, Biomacromolecules 2018, 19, 4122. Fluorescence and photoacoustic images (and spectra) were recorded using the Pearl Impulse Small Animal Imaging System (LI-COR, USA) and Vevo LAZR imaging system (FUJIFILM VisualSonics, Toronto, Canada) with the LZ550 probe (operating frequency of 40 MHz), respectively (see, e.g., A. S. Moses, L. Kadam, A. St Lorenz, M. K. Baldwin, T. Morgan, J. Hebert, Y. Park, H. Lee, A. A. Demessie, T. Korzun, B. Mamnoon, A. W. G. Alani, O. Taratula, L. Myatt, O. R. Taratula, Small 2023, 19, e2202343; and A. S. Moses, O. R. Taratula, H. Lee, F. Luo, T. Grenz, T. Korzun, A. S. Lorenz, F. Y. Sabei, S. Bracha, A. W. G. Alani, O. D. Slayden, O. Taratula, Small 2020, 16, e1906936).

Animals. All animal experiments were approved by the Institutional Animal Care and Use Committee (IACUC) of Oregon Health and Science University and were carried out in accordance with national and local guidelines and regulations (IP00003848). Pregnant CD1 wild-type mice at selected gestational days (Gd) were obtained from Charles River Laboratories (Wilmington, MA).

In Vivo and Ex Vivo Imaging. Pregnant mice at selected Gd6.5 and GD12.5 (3 animals per group, n=3) were injected intravenously (i.v.) via tail vein with NIR-Ps or NIR-SS-Ps (100 μL at 0.3 mg mL-1 SiNc in saline) along with saline as the control group, and biodistribution of NIR dye loaded polymersomes was evaluated in major organs and uterus 24 hours after i.v. injection, using a LI-COR Pearl Impulse Imaging System (LI-COR, Lincoln, NE) with an 800 nm channel, as previously reported (A. S. Moses, L. Kadam, A. St Lorenz, M. K. Baldwin, T. Morgan, J. Hebert, Y. Park, H. Lee, A. A. Demessie, T. Korzun, B. Mamnoon, A. W. G. Alani, O. Taratula, L. Myatt, O. R. Taratula, Small 2023, 19, e2202343). The mean fluorescence intensity of the region of interest was quantified using Pearl Impulse Software. For fluorescence tissue imaging, conceptuses at Gd7.5 and Gd13.5 (left undisturbed in their amniotic sacs) were immediately frozen in cryomolds using liquid nitrogen and optimal cutting temperature (OCT) medium for subsequent sectioning, histology, and fluorescence imaging. The ultrasound/photoacoustic imaging instrumentation (Vevo LAZR, FUJIFILM VisualSonics, Inc., Toronto, Canada) setting used in these studies employs an ultrasound transducer integrated with a tunable pulsed laser operating within 680-970 nm (LZ550), providing real-time acquisition and simultaneous overlay of ultrasound and photoacoustic images on 2D and 3D planes ((A. S. Moses, L. Kadam, A. St Lorenz, M. K. Baldwin, T. Morgan, J. Hebert, Y. Park, H. Lee, A. A. Demessie, T. Korzun, B. Mamnoon, A. W. G. Alani, O. Taratula, L. Myatt, O. R. Taratula, Small 2023, 19, e2202343).

In Vivo MTX-based treatment. The recommended clinical dose for MTX is about 1 mg/kg (or 50 mg/m²). In the first step, to convert human effective dose (HED) in mg/kg to animal (mouse) effective dose (AED) in mg/kg, the following formula was used: AED=HED×12.3 (A. B. Nair, S. Jacob, J. Basic Clin. Pharm. 2016, 7, 27). Thus, AED=1 mg kg-1 dose of MTX×12.3=12.3 mg/kg for mouse. In the next step, to determine experimentally the lowest dose of free MTX and dosage regimen required for complete pregnancy demise, a dose-escalation study was conducted by administering the drug to mice at a range of doses less than 12.3 mg/kg. Briefly, pregnant mice at Gd6.5 (3 animals per group, n=3) were administered a single dose of free MTX (1, 2, 4, or 6 mg/kg) on Gd6.5, and the same dose again on Gd8.5. Mice were allowed to continue gestation after i.v. administration free MTX. Following parturition, pup weights were recorded and analyzed on post-natal day (pnd) 1, 7, 14 and 21. Upon completion of the study, pups were euthanized following an institutionally approved protocol. The results revealed that pregnant mice given 1, 2, and 4 mg/kg of the free drug (2 doses, 2 days apart) continued to grow in size and eventually gave birth to litters, whereas dams given 6 mg/kg free MTX (2 doses) appeared non-pregnant throughout the study and did not give birth to any pups (FIG. 7C). In contrast to free MTX, a dose-escalating study revealed the MTX-SS-Ps formulation was capable of terminating pregnancy at a MTX dose of 1 mg kg-1 (two-dose regimen), i.e., at a dose 6-fold less than that of free MTX (FIG. 7A).

To compare the therapeutic efficacy of different formulations, pregnant mice at Gd6.5 were divided into 6 different groups (n=3): control, free MTX, MTX-Ps, MTX-SS-Ps, empty Ps, and empty SS-Ps. The control groups were injected with saline and empty polymersomes (Ps and SS-Ps). The MTX-treatment groups (free MTX, MTX-Ps, and MTX-SS-Ps) were administered a single dose of 1 mg/kg free MTX or MTX-loaded polymersome formulations (MTX-Ps and MTX-SS-Ps, 1 mg/kg MTX) on Gd6.5, and the same dose again on Gd8.5. Embryonic development in all groups was monitored using a Vevo 2100 high-frequency ultrasound imaging system (Visual Sonics, Toronto, Canada) with an MS-550 high-resolution transducer to obtain images. US images were obtained prior to treatment on Gd6.5, and on Gd8.5, 11.5, and 13.5. The images were analyzed using Vevo LAB software version 5.7.0 and gestational sac lengths were measured. Mice were euthanized on Gd13.5, and uteri were collected for ex vivo imaging and subsequent histological analysis. A board-certified pathologist performed a detailed histopathological assessment of H&E-stained thin sections of fetoplacental units from different treatment groups.

When euthanized at Gd13.5, blood samples were also collected and analyzed by IDEXX Laboratories (Portland, OR, USA) for the total health profile screen to determine plasma levels of cardiac, renal, and hepatic function indicators.

Safety/Toxicity Study. In a separate study, pregnant mice (3 animals per group, n=3) were allowed to continue gestation following i.v. administration of saline, free MTX (1 mg/kg MTX, 2×) or MTX-SS-Ps (1 mg/kg MTX, 2×) on Gd6.5 and Gd8.5. Following parturition, pups were observed to evaluate overall appearance, locomotor activity, feeding, and vocalization. Pup weights were recorded and analyzed on post-natal day (pnd) 1, 7, 14 and 21. Upon completion of the study, pups were euthanized following an institutionally approved protocol. After 2 weeks interval, each female mouse from the treated and control groups was paired with a male for the second breeding. When the pregnant mice gave birth, the pups were monitored and weighed on post-natal day (pnd) 1, 7, 14 and 21 (the same way as for the first pregnancy) to evaluate the safety or toxicity effect of the developed formulations on the subsequent pregnancy. The pups' weights in treatment groups were analyzed and compared to the control groups.

Statistical Analysis. In these studies, no data pre-processing was performed. The data was presented using a mean and standard deviation format (mean+/−SD), with the sample size (n) for each study specified in the figure legends. For comparisons between two groups, a two-tailed unpaired t-test was employed. For more than two groups, one-way analysis of variance (ANOVA) was used to examine the statistical significance. GraphPad Prism v9 (GraphPad Software, CA, USA) was used to perform all statistical analyses.

Redox-triggered release study. MTX drug release from the polymersomes was monitored in the presence of different concentrations of glutathione (GSH) as a function of time (see, e.g., F. Karandish, B. Mamnoon, L. Feng, M. K. Haldar, L. Xia, K. N. Gange, S. You, Y. Choi, K. Sarkar, S. Mallik, Biomacromolecules 2018, 19, 4122). The MTX-loaded polymersomes (10 mg mL-1 of MTX, 1 mL) were placed in Float-A-Lyzer Dialysis tubes (MWCO: 10 kD) floating in a phosphate-buffered saline (PBS) as dialysate either without GSH (pH 7.4) or at 3 different GSH concentrations (5 μM, 1 mM, and 10 mM). At fixed time intervals, 500 μL of dialysate was collected and replaced with 500 μL fresh PBS, and the MTX content within polymersomes was quantified at different time points based on absorption spectra of samples, at 302 nm using high-performance liquid chromatography (HPLC, Shimadzu, Japan) with an Agilent ZORBAX C-18 column (3.5 μm, 4.6×75 mm) at a flow rate of 1 mL min-1 and a mobile phase comprised of acetonitrile/water (35:65 v/v %) stabilized with 0.1% trifluoroacetic acid. The percentage of MTX release at different time points was calculated as follows: Drug release (%)=[MTX]R/[MTX]T×100, where [MTX]R is the amount of MTX released at collection time and [MTX]T is the total amount of MTX that was encapsulated within polymersomes.

Histology and Fluorescence Imaging of Tissues. Fetoplacental tissues were frozen in cryomolds using Tissue-Tek Optimal Cutting Temperature (OCT) compound (Sakura Finetek, Torrence, CA) and liquid nitrogen immediately following euthanasia and necropsy, and stored at −80° C. Tissue thin sections (10 μm) were obtained using a Leica CM 1860 cryostat (Leica Biosystems, Buffalo Grove, IL.) and adhered to Fisherbrand Superfrost Plus microscope slides (Thermo Fisher Scientific, Waltham, MA). Fluorescence micrographs were obtained using a Zeiss Axio Observer 7 equipped with a Plan-Apochromat 20×/0.8 M27 objective and Cy7 reflector (ex: 752/em: 779 nm) while thin sections were still embedded in OCT. Following fluorescence microscopy, the same tissue thin sections were then stained with hematoxylin and eosin (H&E) as follows: (all steps performed at room temperature) 5 min incubation in PBS (pH 7.4) to solubilize OCT, followed by 1 min in Gill's hematoxylin #2, rinsed with tap water, dipped in 2% glacial acetic acid, rinsed again in tap water followed by 2 min incubation with bluing solution and another tap water rinse. Slides were then incubated in 70% ethanol for 5 min, followed by staining with eosin for 1 min, followed by 95%, then 100% ethanol for 5 min, then 2 exchanges of fresh xylene, for 5 min each. Slides were then coverslipped and imaged. Brightfield micrographs of H&E-stained sections were obtained using a Zeiss Axioscan 7 equipped with a Plan-Apochromat 10×/0.45 M27 objective.

### Experimental: Representative Polymersomes and Related Methods for Treating Choriocarcinoma

Materials. PEG(2k)-SS-PCL(5k) (methoxy poly(ethylene glycol)-b-disulfide-poly(ε-caprolactone) copolymer was purchased by Ruixibiotech (Xian, China). NIR dye (silicon 2,3-naphthalocyanine bis(trihexylsilyloxide)) was purchased from Alfa Chemistry (Ronkonkoma, NY, USA). USP-grade methotrexate sodium salt (MTX) was obtained from OHSU Pharmacy (Hikma Pharmaceuticals USA Inc., Berkeley Heights, NJ). 2-Amino-6-chloropurine riboside (6-chloro-guanosine) was purchased from TCI America (Portland, OR). Common chemicals and supplies were provided by MilliporeSigma (Milwaukee, WI), Fisher Scientific Inc. (Hampton, NH), and VWR International, LLC (Radnor, PA).

Computational evaluation of targeted moieties for the choriocarcinoma cell ENT-1 transporter. To identify a useful ligand for binding to the ENT-1 transporter, a group of 20 adenosine analogs from the PubChem database was selected. The ENT-1 crystal structure was generated by the Protein Data Bank (PDB, ID: 6OB7), followed by conducting a molecular docking study using AutoDock Vina. The structure of the ligand molecules was prepared based on their appropriate 3D conformation, while their energy was minimized using PyRx and optimized using Chimera software version 1.17.3. and Modeller v. 10.4 to fix the missing residues of the ENT-1 protein transporter. The protein-ligand complex was visualized by the Biovia Discovery Studio Visualizer.

PEG(2k)-SS-PCL(5k) Modification with Guanosine (Gn).

The conjugation of PCL-SS-PEG-COOH (methoxy poly(ethylene glycol)-b-disulfide-poly(ε-caprolactone)) with 6-chloro-guanosine is illustrated schematically below.

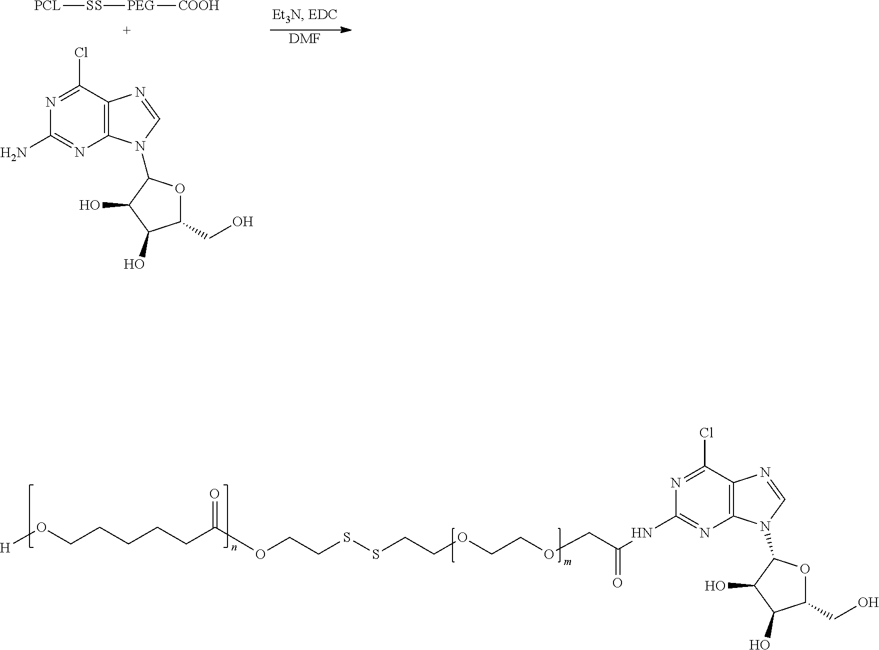

PCL-SS-PEG-COOH polymer (100 mg, 14 μmol, 1 equiv.) dissolved in dimethylformamide (5 ml), 6-chloro-guanosine (5 mg, 17 μmol, 1.2 equiv.) was added. After stirring for 10 minutes, triethylamine (200 μL) and EDC (4 mg, 26 μmol, 1.8 equiv.) were added to the reaction mixture and kept overnight stirring. Next, the reaction mixture was diluted with dichloromethane (25 mL), transferred to a separating funnel, and washed with saturated saline solution three times. The organic layer was collected, dried using anhydrous sodium sulfate, and concentrated using a rotavap. The residue solution was added to chilled ether (−20° C., 45 ml), and the precipitate was collected by centrifugation at 4° C. Trace ether present on the precipitate was removed under vacuum at room temperature. To further remove any impurity, the obtained polymer was dissolved in tetrahydrofuran (THF, 2 ml), loaded on a size exclusion column (Bio-Beads S-X1, Cat. No. 52-2150) and eluted with THE under gravity. The fractions were collected, concentrated under rotavap, precipitated out in chilled ether (−20° C.), dried the precipitate after collecting by centrifugation, and characterized by ¹H NMR. Yield: 98 mg (94%). ¹H NMR (CDCl₃, 400 MHz): δ 8.08-8.11 (m, 2H), δ 6.66-6.67 (t, 2H), δ 5.51-5.53 (m, 8H), δ 4.16-4.30 (m, 6H), δ 3.93-4.05 (m, 94H), δ 3.87-3.99 (m, 22H), δ 3.54-3.57 (m, 181H), δ 3.17 (s, 6H), δ 3.07 (s, 2H), δ 2.74-2.92 (m, 10H), δ 2.20-2.31 (m, 96H), δ 1.53-1.65 (m, 192H), δ 1.27-1.35 (m, 94H). The conjugation of PCL-SS-PEG-COOH with 6-chloro-guanosine, which exists as α-form and β-form (anomers), leads to the formation of a mixture of two isomeric products. This has been confirmed by the observation of two protons in the NMR peaks that correspond to that of conjugated 6-chloro-guanosine.

To evaluate the conjugation yield of Gn to polymer, the Pierce Quantitative Fluorometric Peptide Assay kit (Thermo Scientific, Rockford, IL) was used to measure the free amine groups before filtering the reaction mixture based on the manufacturer's recommendation. According to the amount of the initial polymer used for the polymersome preparation, the yield of Gn-conjugated PEG-SS-PCL was determined to be 99%.

Polymersome preparation. MTX-loaded polymersomes were prepared using the NanoAssemblr™ Ignite™ microfluidic mixer (Precision NanoSystems). First, methotrexate sodium salt (30 mg) was dissolved in 1 mL saline (syringe 1). To prepare non-targeted nanoparticles, PEG-SS-PCL polymer (M.W.=7 kDa, 10 mg, RuixiBiotech, China) was added to 1 mL acetone (syringe 2). Targeted polymersome samples were prepared using 9 mg of the PEG-SS-PCL polymer and 1 mg of the conjugated PCL-SS-PEG-Gn polymer (10%) in 1 mL acetone (syringe 2). The syringes were installed into the NanoAssemblr™ Ignite™ Ignite platform through a NxGen cartridge (Precision Nanosystems). The organic and aqueous solutions were run at 1:1 flow ratio followed by 9 mL/min total flow rate. The polymersome sample mixture was then collected in a glass vial and bubbled air to remove the organic phase. Finally, the sample was centrifuged using an Amicon Ultra-4 centrifugal filter tube (MWCO: 30 kDa, 10 min), and the top solution was collected and passed through 0.2-micron filter. For NIR Imaging, NIR dye (naphthalocyanine derivative) was loaded into polymersomes using a glass microfluidic mixer chip (Precigenome, San Jose, CA) compatible with THF: copolymer (10.0 mg, PEG-SS-PCL) and NIR dye (0.3 mg) were dissolved in tetrahydrofuran (THF, 1 mL total) to generate NIR-SS-Ps.

Characterization of nanoparticles. The polymersomes' size, surface charge, and polydispersity were determined using a Malvern ZetaSizer (Worcestershire, UK). Transmission Electron Microscopy (TEM) images were acquired on JEOL JEM-2100 LB6 (Peabody Massachusetts) at North Dakota State University (NDSU) Electron Microscopy Core to assess the morphology of polymersomes. This material is based upon work supported by the National Science Foundation under Grant No. 0821655. The loading of MTX into the polymersome nanoparticles was evaluated using high-performance liquid chromatography (HPLC, Shimadzu, Japan) at a wavelength of 302 nm. The analysis was performed with an Agilent ZORBAX C-18 column (3.5 μm, 4.6×75 mm), operating at a flow rate of 1 mL/min. The mobile phase consisted of acetonitrile/water (35:65 v/v) with 0.1% trifluoroacetic acid.

Animals. All animal experiments were approved by the Institutional Animal Care and Use Committee (IACUC) of Oregon Health and Science University and were carried out in accordance with national and local guidelines and regulations (IP00000033). Athymic nude mice were obtained from Charles River Laboratories (Wilmington, MA).

Biodistribution study. Choriocarcinoma tumor-bearing mice (3 animals per group, n=3) were injected intravenously (i.v.) via tail vein with NIR@SS-Ps or Gn-NIR@SS-Ps (100 μL at 0.3 mg/mL SiNc in saline) as non-targeted and targeted polymersomes, respectively, along with saline as the control group, and biodistribution of NIR dye loaded polymersomes was evaluated in major organs and tumors 12 hours after i.v. injection, using a LI-COR Pearl Impulse Imaging System (LI-COR, Lincoln, NE) with an 800 nm channel. The mean fluorescence intensity of the region of interest was quantified using Pearl Impulse Software.

Anti-tumor Efficacy. Choriocarcinoma tumor-bearing mice were divided into 4 different groups (3 animals per group, n=3): control (saline), free MTX, MTX@SS-Ps, and Gn-MTX@SS-Ps. The MTX-treatment groups (free MTX, MTX@SS-Ps, and Gn-MTX@SS-Ps) were administered 10 mg/kg dose of free MTX or an equivalent dose of MTX-loaded polymersomes (MTX@SS-Ps and Gn-MTX@SS-Ps) on days 6, 8, 10, 13, 15, and 17 after tumor inoculation. Length and width of tumors were measured using calipers for 10 days following treatment and used to calculate tumor volume as V=W (2)×L/2, where V, W, and L are volume, width, and length of the tumor, respectively. Mice were euthanized on day 23.

Safety evaluation of MTX-loaded polymersomes. In a separate study, 2 groups of mice were treated with saline (control) and MTX@SS-Ps nanoparticles (treatment group, 10 mg/kg) every other day for two weeks (total 6 injections). Body weight was recorded every other day. At the end of week 3, the mice were euthanized and their whole blood was collected and submitted to IDEXX Laboratories for a total Health Profile screen to determine plasma levels of liver, renal, and cardiac function indicators, including creatinine, blood urea nitrogen (BUN), aspartate aminotransferase (AST), alkaline phosphatase (ALP), creatine kinase (CK), alanine transaminase (ALT), albumin, and globulin.

Statistical Analysis. All data processing was conducted using GraphPad Prism v10 (GraphPad Software, CA, USA) and results were expressed as the mean±SD. The sample size (n) for each study is specified in the figure legends. A two-tailed unpaired t-test was used for comparisons between two groups, while one-way analysis of variance (ANOVA) was applied for comparisons among more than two groups. The statistical significance threshold was measured as P<0.05 (*p<0.05, **p<0.01, ***p<0.001, ****p<0.0001).

As used herein, the term “about” refers to ±5% of the specified value.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for treating an ectopic pregnancy, comprising administering in a single dose a therapeutically effective amount of a methotrexate-containing polymersome to a subject in need thereof,
wherein the methotrexate-containing polymersome comprises methotrexate and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block, and
wherein methotrexate, or a pharmaceutically acceptable salt thereof, is encapsulated in the hydrophilic interior core.

2. The method of claim 1, wherein the methotrexate-containing polymersome contains from about 20 to about 50 mg methotrexate per 10 mg polymersome.

3. The method of claim 1, wherein the amount of methotrexate administered is from about 1 mg/m² to about 50 mg/m² (methotrexate/subject surface area).

4. The method of claim 1, wherein the amount of methotrexate administered is from about 0.1 mg/kg to about 2.0 mg/kg (methotrexate/subject body weight).

5. The method of claim 1, wherein the methotrexate-containing polymersome is administered by intravenous injection.

6. A methotrexate-containing polymersome, comprising:
(a) methotrexate, or a pharmaceutically acceptable salt thereof, and
(b) an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block, and
wherein methotrexate, or a pharmaceutically acceptable salt thereof, is encapsulated in the hydrophilic interior core.

7. The polymersome of claim 6, wherein hydrophilic PEG block has a molecule weight from about 2 kDa to about 10 kDa.

8. The polymersome of claim 6, wherein hydrophobic PCL block has a molecule weight from about 5 kDa to about 20 kDa.

9. The polymersome of claim 6, wherein methotrexate is present in the polymersome in an amount from about 35 mg per 47 mg polymersome or in an amount from about 0.75 mg per 1.0 mg polymersome.

10. The polymersome of claim 6, wherein methotrexate is present in the polymersome in an amount of about 0.75 mg per 1.0 mg polymersome.

11. A pharmaceutical composition comprising the polymersome of claim 6 and a pharmaceutically acceptable carrier.

12. The polymersome of claim 6 in lyophilized form.

13. A method for making the polymersome of claim 6, comprising microfluidic mixing of a first solution of an amphiphilic block polyethylene glycol-disulfide-polycaprolactone polymer and a second solution of methotrexate, wherein the first and second solutions are water miscible.

14. The method of claim 1, wherein the amount of methotrexate is sufficient to result in resolution of serum human chorionic gonadotropin.

15. The method of claim 1, wherein the amount of the methotrexate administered from about 0.9 mg/kg to about 1.1 mg/kg (methotrexate/subject body weight).

16. The method of claim 1, wherein the amount of the methotrexate administered is about 50 mg/m² (methotrexate/subject surface area).

17. The polymersome of claim 6 further comprising a targeting agent.

18. The polymersome of claim 17, wherein the targeting agent is a guanosine.

19. A method for treating gestational trophoblastic neoplasia (GTN) in a subject, comprising administering to a subject in need thereof, a therapeutically effective amount of a methotrexate-containing polymersome,
wherein the methotrexate-containing polymersome comprises methotrexate and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block,
wherein methotrexate, or a pharmaceutically acceptable salt thereof, is encapsulated in the hydrophilic interior core, and
wherein the polymersome comprises one or more guanosine moieties to target equilibrative nucleoside transporter 1 (ENT-1) expressed on the surface of the neoplasia cells.

20. A method for treating gestational choriocarcinoma in a subject, comprising administering to a subject in need thereof a therapeutically effective amount of a methotrexate-containing polymersome,
wherein the methotrexate-containing polymersome comprises methotrexate and an amphiphilic block polyethylene glycol-disulfide-polycaprolactone (PEG-SS-PCL) copolymer having a hydrophilic PEG block and a hydrophobic PCL block with a disulfide moiety covalently coupling the PEG block to the PCL block,
wherein the polymersome has a bilayer structure, with a hydrophilic interior core, hydrophobic layer and a hydrophilic outer shell formed by the hydrophilic PEG block,
wherein methotrexate, or a pharmaceutically acceptable salt thereof, is encapsulated in the hydrophilic interior core, and
wherein the polymersome comprises one or more guanosine moieties to target equilibrative nucleoside transporter 1 (ENT-1) expressed on the surface of choriocarcinoma cells.
